SEQUENCE LISTING (15 sequences)

SEQ ID NO: 1 - protein, 28 aa, Artificial sequence
  Synthetic peptide
AYARAAARQARAVGFPVTPQVPLRPMTY

SEQ ID NO: 2 - protein, 28 aa, Artificial sequence
  Synthetic peptide
AYARAAARQARAVGFPVTPQVPARPMTY

SEQ ID NO: 3 - protein, 33 aa, Artificial sequence
  Synthetic peptide
ARQIKIWFQNRRMKWKKVGFPVTPQVPLRPMTY

SEQ ID NO: 4 - protein, 33 aa, Artificial sequence
  Synthetic peptide
ARQIKIWFQNRRMKWKKVGFPVTPQVPARPMTY

SEQ ID NO: 5 - protein, 24 aa, Artificial sequence
  Synthetic peptide
ARRMKWKKVGFPVTPQVPLRPMTY

SEQ ID NO: 6 - protein, 24 aa, Artificial sequence
  Synthetic peptide
ARRMKWKKVGFPVTPQVPARPMTY

SEQ ID NO: 7 - protein, 16 aa, Artificial sequence
  Synthetic peptide
IGVSATPKLPLRAISR

SEQ ID NO: 8 - protein, 16 aa, Artificial sequence
  Synthetic peptide
VGFYVKPRTPLRELAH

SEQ ID NO: 9 - protein, 16 aa, Artificial sequence
  Synthetic peptide
VGFRVAPNNPLRTMTF

SEQ ID NO: 10 - protein, 16 aa, Artificial sequence
  Synthetic peptide
VGFAVMPGVPLRSMTY

SEQ ID NO: 11 - protein, 16 aa, Artificial sequence
  Synthetic peptide
VGFPVWPCVPLRPMTY

SEQ ID NO: 12 - protein, 16 aa, Artificial sequence
  Synthetic peptide
VGFPVSPQVPLRIMTY

SEQ ID NO: 13 - protein, 16 aa, Artificial sequence
  Synthetic peptide
VGFPVHPQVPLRQMTY

SEQ ID NO: 14 - protein, 16 aa, Artificial sequence
  Synthetic peptide
VGFPVQPQVPLRQMTY

SEQ ID NO: 15 - protein, 16 aa, Artificial sequence
  Synthetic peptide
VGFPVCPQVPLRQMTY

(19) United States
(12) Patent Application Publication
(10) Pub. No.: US 2003/0229906 A1
(43) Pub. Date: Dec. 11, 2003

(54) Methods and compositions for the treatment of disorders of HIV infection

(76) Inventors: Irwin H. Gelman, Buffalo, NY (US); Paul Klotman, New York, NY (US); Ming Ming Zhou, Old Greenwich, CT (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/413,785

(22) Filed: Apr. 14, 2003

Related U.S. Application Data
(60) Provisional application No. 60/372,557, filed on Apr. 15, 2002.

(51) Int. Cl. A01K 67/00; C12Q 1/70; A61K 39/395; C12N 15/867

(52) U.S. Cl. 800/8; 424/145.1; 435/5; 435/325; 435/456

(57) ABSTRACT

The present invention relates to methods and compositions for use in the intervention of diseases associated with HIV infection. In exemplary embodiments, methods and compositions for the treatment of HIV associated nephropathy (HIVAN) are disclosed.

The present application claims the benefit of priority of U.S. Provisional Application No. 60/372,557, which was filed on Apr. 15, 2002 and is incorporated herein by reference in its entirety.

### FIELD OF THE INVENTION

The present invention generally relates to the treatment or inhibition of diseases associated with HIV-1 infection. In particular, the present invention provides methods and compositions for decreasing, inhibiting, or otherwise abrogating the interaction of Nef with a SH3 domain of a Src family tyrosine kinase.

### BACKGROUND OF THE INVENTION

Due to the introduction of triple-drug combination antiretroviral therapy, the morbidity and mortality in symptomatic and asymptomatic human immunodeficiency virus type 1 (HIV) infected individuals has decreased markedly (Hammer et al., N Engl J Med., 337:725-733, 1997; Cameron et al., Lancet, 351:543-549, 1998; Montaner et al., JAMA, 279:930-937, 1998). As a result, triple-drug regimens have been widely adopted for the treatment of HIV infection starting in 1996 (Carpenter et al., JAMA, 283:381-390, 2000; Gazzard et al., Lancet, 1998;352:314-316, 1998; Guidelines for the Use of Antiretroviral Agents in HIV-Infected Adults and Adolescents. Washington, D.C.: US Dept of Health and Human Services/Henry J. Kaiser Family Foundation; January 2000). Through the use of powerful triple-drug cocktails, the prognosis for HIV-infected patients has improved markedly.

Unfortunately, along with the increased life-expectancy of HIV-infected patients, these patients increasingly develop diseases associated with prolonged HIV-1 infection. These diseases seem to result from the expression of HIV-encoded proteins in non-lymphoid tissues, even in the absence of ongoing viral replication. Thus, as HIV infection is being turned from predictable AIDS into a maintenance disease, the new challenge for clinicians becomes a question of controlling the emergence of these HIV-induced diseases. One such disease is HIV-associated nephropathy (HIVAN); this is a progressive glomerular and tubular disease that is increasingly common in AIDS patients (Bruggeman et al., J. Clin. Invest. 100(1):84-92, 1997; Rao, Semin Nephrol. 18:378-395, 1998; Bourgoignie et al., Kidney Int Suppl. 35:S19-23, 1991). It is the most common cause of chronic renal disease in HIV-1 infected patients and affects mostly African Americans (Winston et al., Semin. Nephrol. 20(3)293-8, 2000).

HIVAN is characterized by proteinuria, rapidly developing azotemia and histologically by collapsing variant of focal and segmental glomerulosclerosis with acute tubular necrosis (Rajvanshi et al., J Assoc Physicians India, 49:813-8, 2001) and proliferation of renal tubular, parietal, and visceral epithelial cells (podocytes). In addition, the disease manifests in tubulointerstitial infiltration with mononuclear cells, edema, fibrosis, and microcystic tubule dilation (D'Agati et al., Kidney Int. 35:1358-1370, 1989; Cohen et al., Mod Pathol. 1:87-97, 1988). Untreated, it may result in end stage renal disease (ESRD) in as little as four months and is the third leading cause of ESRD in blacks age 20 to 64 (Monahan et al., Semin Nephrol., 21(4):394-402, 2001). The incidence of HIVAN continues to increase and is the single most common cause of chronic renal disease in HIV-1 seropositive patients. Improvements in survival rates of HIV-1-seropositive patients on hemodialysis and improved treatment of HIV with highly active antiretroviral therapy (HAART) and angiotensin-converting enzyme (ACE)-inhibitors will result in an increased prevalence of HIVAN in ESRD and pre-ESRD patient populations. Thus, left unchecked HIVAN promises to become an urban epidemic as anti-HIV treatments prolong the lives of HIV-infected patients.

Expression of HIV-1 mRNA in tubular and glomerular epithelial cells in biopsies from HIVAN patients, as well as in these cell types in transgenic (Tg) mouse model of HIVAN (Bruggeman et al., J Clin Invest. 100:84-92, 1997; Bruggeman et al., J Am Soc Nephrol. 11:2079-2087, 2000), strongly suggests that HIV-1 mRNA expression in these sites contributes to the disease process. In both the murine model and human renal biopsy material, one of the prominent pathological characteristics of HIVAN is the hyperproliferation of podocytes manifested by expression of the cell cycle proliferation marker Ki-67 and the loss of differentiation markers synaptopodin, WT-1, GLEPP-1, and CALLA (Barisoni et al., J Am Soc Nephrol 10:51-61, 1999). Although the renal epithelial cells appear to be the main target for HIV-1 pathogenesis, the HIV gene products responsible for tissue-specific renal pathology remain unknown.

HIV-1 encodes three structural genes (gag, pol, env), two essential regulatory genes (tat and rev), and four accessory genes (vif, vpr, vpu and nef; Frankel et al., Annu Rev Biochem 67:1-25, 1998). An HIV-1 plasmid construct deleted for gag and pol (pNL4-3: d1443) has been used to generate a transgenic mouse model (Dickie et al., Virology 185:109-119, 1991; Kopp et al., Contrib Nephrol 107:194-204, 1994). These animals present with renal disease that is clinically and pathologically identical to that observed in patients with HIVAN. Thus, this construct expressing Env and the accessory proteins (Vif, Vpr, Vpu, Nef, Tat, and Rev) without Gag/Pol can induce HIVAN. Previously, the lack of an in vitro podocyte culture system prevented a detailed analysis of the effects of HIV-1 gene expression on renal podocytes. Conditionally immortalized murine podocytes also are available (Mundel et al., Exp Cell Res 236:248-258, 1997; Schwartz et al., J Am Soc Nephrol 12:1677-1684., 2001). However, despite the availability of this in vitro cell line and a transgenic model which expresses Env and the accessory proteins, Vif, Vpr, Vpu, Nef, Tat, and Rev but not Gag/Pol, the pathological cause of HIVAN remains unknown.

Thus, there is a need to identify the cause of HIVAN so that treatment regimens can be designed to ameliorate secondary disorders associated with HIV-infection.

### SUMMARY OF THE INVENTION

The present invention provides methods of inhibiting kidney cell dedifferentiation, comprising inhibiting the interaction of Nef with a Src family tyrosine kinase SH3 domain of a polypeptide of the cell. Such a cell may be located in vitro or in vivo. In preferred embodiments, the Nef is HIV-1 Nef. In particularly preferred embodiments, the kidney cell is a podocyte.

In general terms, inhibiting the interaction of Nef with a SH3 domain of a Src family tyrosine kinase comprises reducing the expression of Nef in the cell. In certain embodiments in which it is desirable to reduce the expression of Nef, the method comprises contacting the cell with a nucleic acid construct that reduces the expression of Nef in the cell.

In other aspects, inhibiting the interaction of Nef with a SH3 domain of a Src family tyrosine kinase comprises contacting the Nef with an agent that binds to and/or inactivates the Nef. In specific embodiments, the agent may be a peptide inhibitor comprising a variant of the PXXP motif of the SH3 binding domain of Nef. Exemplary peptide inhibitors may comprise a sequence selected from the group consisting of SEQ ID NO:1, SEQ ID NO:2, SEQ ID NO:3, SEQ 4, SEQ ID NO:5, SEQ ID NO: 6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, and SEQ ID NO:15. Of course it should be understood that these inhibitors are merely exemplary and given the teachings of the present invention, those of skill in the art will be able to design other peptide inhibitors that binds to and/or inactivates the Nef.

In certain examples of the present invention, the agent may be a small molecule antagonist of the SH3 binding domain of Nef. Also contemplated are embodiments in which the agent is a peptidomimetic antagonist of the SH3 binding domain of Nef. In alternative embodiments, the agent may be an anti-Nef antibody preparation. Such an antibody preparation may comprise a single chain antibody. Alternatively, such an antibody may be a monoclonal antibody. Preferably, the monoclonal antibody is one which binds the PXXP motif of the SH3 binding domain of HIV-1 Nef.

Another aspect of the present invention describes a transgenic non-human animal, wherein a podocyte of the animal comprises an HIV-1 Nef gene under the control of a kidney cell-specific promoter. In particular embodiments, the promoter is a nephrin promoter. In alternative embodiments, the promoter is a CX promoter. Preferably, the specific activity of a Src family tyrosine kinase in the podocyte of the transgenic animal is increased relative to the Src tyrosine kinase activity level of a podocyte from a wild-type animal of the same species.

In preferred embodiments, the expression of one or more nucleic acids selected from the group consisting of Cek 5 receptor protein tyrosine kinase ligand; Cyclin dependent kinase inhibitor p57; interleukin-5 receptor; nucleobindin; Heat shock transcription factor 1; erythrocyte glucose transporter-1 (GLUT-1); monocyte chemoattractant protein 1 receptor (CCR2); hepatocyte nuclear factor 3; pur-alpha; CTCF; UBF; Ski proto-oncogene; Sp4 transcription factor; transforming growth factor beta; xeroderma pigmentosum group B complementing protein (XPB); cyclin B1; Integrin beta; Egr-1; c-erbA; Tob (Transducer of ErbB-2); xeroderma pigmentosum group G complementing protein (XPG); and granulocyte-macrophage colony stimulating factor receptor is decreased in a podocyte of the transgenic animal relative to a podocyte from a wild-type animal of the same species. Alternatively, the transgenic animal is one in which the expression of one or more nucleic acids selected from the group consisting of Hox-2.5; clusterin; cyclin B2; PCNA; HMG-14 chromosomal protein; and B-Raf proto-oncogene is increased in a podocyte of the transgenic animal relative to a podocyte from a wild-type animal of the same species.

The present invention further contemplates methods and compositions for making and using a recombinant host cell, wherein the cell is transformed with an expression construct comprising a nucleic acid that encodes HIV-1 Nef under the control of a kidney cell-specific promoter. In specific embodiments, the cell is a podocyte. Preferably, the promoter is a nephrin promoter. It should be noted that while the nephrin promoter is indicated as a preferred example, any kidney cell specific promoter may be used in the present invention. In specific aspects of the present invention, the expression construct comprises a Nef sequence from pNL4-3 contained in GenBank Accession # AF324493 (nucleotides 8787 to 9407).

Another aspect of the present invention is directed to a method for screening for agents that modulate nephropathy comprising:

a) providing a cell expressing HIV-1 Nef;

b) contacting the cell with a candidate modulator; and

c) monitoring the cell for change in a cellular property associated with nephropathy that occurs in the presence of the modulator. Preferably the cell is a kidney cell and more specifically, the cell is a podocyte. The podocyte may be a primary podocyte. Alternatively, the podocyte may be derived from a cell line. An exemplary primary podocyte that may be used in the method is one which is derived from a subject having HIVAN, however it need not be derived from such a subject. In the screening methods described herein, the contacting may be performed in vitro or in vivo. Indeed, the screening methods may be performed at different levels, where an initial screen involves an in vitro screen followed by a subsequent in vivo screening step.

The monitoring for the screening assay may comprise monitoring the specific activity of Src family tyrosine kinases of the cell in the presence and absence of the candidate modulator. Alternatively, the monitoring may comprise determining the expression of more one or more nucleic acids selected from the group consisting of Cek 5 receptor protein tyrosine kinase ligand; Cyclin dependent kinase inhibitor p57; interleukin-5 receptor; nucleobindin; Heat shock transcription factor 1; erythrocyte glucose transporter-1 (GLUT-1); monocyte chemoattractant protein 1 receptor (CCR2); hepatocyte nuclear factor 3; pur-alpha; CTCF; UBF; Ski proto-oncogene; Sp4 transcription factor; transforming growth factor beta; xeroderma pigmentosum group B complementing protein (XPB); cyclin B1; Integrin beta; Egr-1; c-erbA; Tob (Transducer of ErbB-2); xeroderma pigmentosum group G complementing protein (XPG); granulocyte-macrophage colony stimulating factor receptor; Hox-2.5; clusterin; cyclin B2; PCNA; HMG-14 chromosomal protein; and B-Raf proto-oncogene in the presence and absence of the candidate modulator.

In particular embodiments, the cell is part of a transgenic, non-human animal. In the in vivo screens, a readout for the screen may involve monitoring protein excretion of the animal.

In specific aspects, the candidate modulator may be a nucleic acid construct that reduces the expression of Nef. Alternatively, the candidate modulator is an antibody (e.g., a single chain antibody or a monoclonal antibody). In particular aspects, the monoclonal antibody binds the PXXP motif of the SH3 binding domain of HIV-1 Nef.

The present invention further contemplates a candidate modulator of nephropathy identified according to the screening methods of the present invention. Such a modulator may be formulated into a pharmaceutical composition.

The present invention also contemplates a peptide composition comprising a sequence selected from the group consisting of the peptide sequences described in Table 1 and Table 1A.

Also described is a method of treating a subject, comprising inhibiting the interaction of Nef with a SH3 domain of a Src family tyrosine kinase, wherein the subject has a disease associated with HIV-1 infection. Exemplary diseases associated HIV-1 infection include but are not limited to a HIV-induced disease selected from the group consisting of HIV associated nephropathy (HIVAN) AIDS dementia; anemia; lymphoma; myopathy; cardiomyopathy; and primary HIV-induced disease progression. Contemplated treatment methods include administering compositions identified according to the present invention, either alone and/or in combination with other anti-HIV treatments.

In addition to the foregoing, the invention includes, as an additional aspect, all embodiments of the invention narrower in scope in any way than the variations specifically mentioned above. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

Also, it should be understood that the detailed description presented below, while providing preferred embodiments of the invention, is intended to be illustrative only since changes and modification within the scope of the invention will be possible whilst still providing an embodiment that is within the spirit of the invention as a whole.

### BRIEF DESCRIPTION OF THE DRAWINGS

The following drawings form part of the present specification and are included to further demonstrate aspects of the present invention. The invention may be better understood by reference to the drawings in combination with the detailed description of the specific embodiments presented herein.

FIG. 1. Schematic representation of HIV-1 provirus (pNL4-3), gag/pol deletion constructs (pNL4-3:d1443) and pNL4-3: ΔG/P-EGFP in which gag/pol sequence region was substituted with EGFP gene at Sph I and Msc I sites.

FIG. 2A through FIG. 2B. Colony formation in soft agar by podocytes transduced with the parental virus (NL4-3: ΔG/P-EGFP) or control virus (HR-CMV-IRES2-EGFP): Confluent monolayer of podocytes on plastic plate transduced with parental virus (FIG. 2A) or control virus (FIG. 2D) observed by fluorescence microscopy (Olympus I×70). Colony formation by podocytes transduced with parental virus was observed in soft agar as viewed under bright light (FIG. 2B) or fluorescence microscopy (FIG. 2C) after 4 weeks of incubation. Colony formation in soft agar was not observed with the control virus as viewed under bright light (FIG. 2E) or fluorescence microscopy (FIG. 2F). The colonies were viewed under 10× objective.

FIG. 3A through FIG. 3F. Representative colony formation activity in soft agar by podocytes transduced with the Env, Vif and Nef deleted viruses: FIG. 3A, FIG. 3B, FIG. 3C under bright light and FIG. 3D, FIG. 3E, FIG. 3F under fluorescent light represent Env, Vif and Nef respectively.

FIG. 4A through FIG. 4B. Expression of Nef in pBabe-puro retroviral expression vector (FIG. 4A) and colony formation analysis of podocytes 4 weeks after incubation (FIG. 4B). FIG. 4A; western blot showing expression of Nef in 293T cells and podocytes transduced with NL4-3: ΔG/P-EGFP virus (lanes 1 and 2 respectively), podocytes transduced with control Babe-puro virus (lane 3), and podocytes transduced with Babe-puro/Nef virus (lane 4). FIG. 4B; anchorage-independent growth of podocytes transduced with control Babe-puro virus (a), and Babe-puro/Nef virus (b). The presence of Nef clearly demonstrates the induction of anchorage-independent growth. The colonies were viewed under 10× objective (Olympus I×70).

FIG. 5. Quantification of colony formation by podocytes transduced with gag/pol deleted parental HIV-1, mutated viral constructs, Babe-puro/Nef construct or control empty vectors. After transduction, 40,000 cells were incubated in soft agar and colonies were counted after 4 weeks of incubation. The experiments were repeated three times for each construct in triplicate plates. An average of colony formation per plate was taken to calculate the percentage of colony forming frequency. Variability was consistently less than 10% within each experiment.

FIG. 6. Graph showing growth of podocytes expressing Nef (—●—) or vector alone (---◯---) in cell culture for 15 days under permissive conditions. Initially 10,000 cells suspended in 1.0 ml growth medium were seeded in 24-well plate. The cells were counted at 3-day intervals in quadruplicate wells after trypan blue dye exclusion. The mean of cells per well ±SD was plotted for each group of cells. The arrow indicates the time at which cells reached to confluence. No statistically significant difference in cell count was observed before confluence (P>0.47) while it was significant after confluence (P<0.001).

FIG. 7. Podocytes infected with pBabe-Puro vector alone (FIGS. 7A and 7C) or pBabe-Puro/Nef (FIG. 7B and FIG. 7D). Upper panel (FIG. 7A and FIG. 7B), the cells stained with Wright Giemsa stain after 10 days of incubation at permissible temperature, and the lower panel (FIG. 7C and FIG. 7D), the same cells observed under light microscope with 10× objective (Olympus 1×70). The Nef expressing podocytes form foci whereas podocytes with vector alone show a monolayer with distinct boundaries.

FIG. 8. Effect of HIV-1 infection on podocytes. Podocytes transduced with HIV-1 pNL4-3:d1443(HIV) and mock-transduced podocytes (Control) were compared. Gene expression was analyzed by northern blot. 10 μg of total RNA was loaded in each lane, and a G3PDH probe was used as a control.

FIG. 9A through FIG. 9C. FIG. 9A. Synaptopodin expression in podocytes transduced with viruses mutated in env, nef, rev, vif, vpr, or vpu. Podocytes were infected with HIV-1 NL4-3 (HIV-1), ΔEnv, ΔNef, ΔRev, ΔVif, ΔVpr, ΔVpu, Tat single gene construct (Tat), or vector alone (Vector) viruses, and then were cultured under non-permissive conditions for 14 days. The expression of synaptopodin was analyzed by northern blot. Blots representative two independent experiments. FIG. 9B Northern blots were quantitated and normalized to G3PDH. The level of expression relative to Mock control (fold induction) is indicated. Bars represent mean±SD of two experiments. FIG. 9C Effect of Nef or Vif on synaptopodin expression. Podocytes were transduced with viruses and were cultured under non-permissive conditions for 14 days without puromycin. The expression of synaptopodin was analyzed by northern blot analysis. pHR-CMV-IRES2-GFP-ΔB vector (pHR vector), HIV-1 pNL4-3 (HIV), pBabe-puro expression vector (pBabe vector), pBabe-puro/nef(Nef), pHR-CMV-IRES2-GFP-ΔB/vif(Vif).

FIG. 10A through FIG. 10B. FIG. 10A Effect of Nef on gene expression. Podocytes were transduced with pBabe-puro/nef(Nef) or pBabe-puro expression vector (Vector). Cells were cultured under non-permissive conditions for 14 days and gene expression was analyzed by northern blot. FIG. 10B. Effect of Tat on gene expression. Podocytes were transduced with pHR-CMV-IRES2-GFP-ΔB/tat (Tat) or pHR-CMV-IRES2-GFP-ΔB vector (Vector). Cells were cultured under non-permissive conditions for 14 days and gene expression was analyzed by northern blot.

FIG. 11A through FIG. 11B Morphology of podocytes. FIG. 11A. Puromycin-selected podocytes transduced with pBabe-puro vector (Vector). FIG. 11B. Puromycin-selected podocytes transduced with pBabe-puro/nef (Ne). Podocytes were cultured for 14 days under nonpermissive conditions in the absence of puromycin. Original magnification, ×200.

FIG. 12 Total number of podocytes transduced with pBabe-puro expression vector (Vector) or pBabe-puro/nef (Nef). All cells were plated in collagen-coated 6-well plates at 20,000 cells/well, and were cultured for 7 or 14 days under nonpermissive conditions without puromycin. Bars represent mean±SD of triplicate samples.

FIG. 13 Src and Hck tyrosine kinase activity. Podocytes transduced with Nef or vector were cultured under nonpermissive conditions for 14 days. RIPA lysates were immunoprecipitated with anti-Src or anti-Hck antibody, and were incubated with [γ-³²P]-ATP and enolase. The Hck autophosphorylation band was verified after autoradiography by directing immunoblotting with anti-Hck and chemiluminescence analysis. Lysates were also analyzed by western blotting for the expression of Src and Hck.

### DETAILED DESCRIPTION OF THE PREFERRED EMBODIMENTS OF THE INVENTION

The prognosis for HIV-infected patients has improved markedly with the introduction of antiviral cocktail therapies. However, even in the absence of HIV viral replication, the expression of HIV-encoded proteins in the non-lymphoid tissues of the patient result in secondary diseases associated with HIV infection. Such diseases include, but are not limited to HIVAN, AIDS dementia; HIV-induced anemia; HIV-induced lymphoma; HIV-induced myopathy; HIV-induced cardiomyopathy; and primary HIV-induced disease progression. Thus, while clinicians are able to control the HIV replication, many patients die having succumbed to these secondary diseases.

The present invention, for the first time shows HIV-associated diseases are associated with the expression of Nef and its ability to activate Src family tyrosine kinases. For example, the present invention shows that there is an HIV-1-mediated dysregulation of the podocyte phenotype in collapsing glomerulopathy as a result of altered expression of differentiation markers in podocytes. Further, it is demonstrated herein that expression of HIV-1 Nef in podocytes is necessary and sufficient for the phenotypic changes associated with HIVAN, a major secondary disease associated with HIV infection.

The present invention shows that Nef is responsible for the phenotypic changes observed in HIVAN. For example, at the molecular level, p57 was found to be downregulated nearly 4-fold by Nef. Ezrin, which is expressed in normal podocytes and diminishes in an early stage of podocyte injury (Hugo et al., Kidney Int. 54:1934-1944, 1998), was decreased by Nef The expression of PCNA, clusterin, and b-Raf was increased in Nef-transduced podocytes. In contrast, the expression of p57, hepatocyte nuclear factor 3, pur-alpha, CTCF, c-erb A, and Tob was decreased by Nef. At the morphological level, Nef enhanced the proliferation of podocytes, and is shown to be responsible for the loss of contact inhibition observed in HIV-infected podocytes. The present invention further demonstrates that Nef causes these phenotypic changes through its interaction with Src family tyrosine kinases via it tyrosine kinase SH3 binding domain.

In light of the above findings, the present invention provides methods and compositions for the treatment disorders associated with HIV infection. In particular, the methods and compositions are designed to disrupt, inhibit, decrease or otherwise abrogate the interaction of Nef with the SH3 domain of Src family tyrosine kinases. Such methods and compositions antagonize Nef's ability to activate proliferation and dedifferentiation signaling mediated by the Src family tyrosine kinases. The invention includes methods of making and using peptides, small molecule inhibitors, nucleic acid-based therapies either alone or in combination with other known therapeutic interventions for HIV-based disorders. It should be noted that while the Examples of the present invention are written with respect to HIVAN, the methods and compositions of the present invention may be used in the therapeutic intervention of any disorder associated with HIV infection that is mediated through a Nef/Src interaction. The methods and compositions of the present invention are described in further detail herein below.

A. Nef/Src Interaction

The Nef peptide encoded by HIV-1 is well known to those of skill in the art (see e.g., U.S. Pat. No. 5,705,612; U.S. Pat. No. 5,968,514, U.S. Pat. No. 6,261,564). This protein is a highly conserved 27-34 kDa myristoylated HIV-1 protein that is one of a set of accessory proteins characteristic of primate lentiviruses (Foti et al., J. Biol. Chem., 274(49):34765-34772, 1999). It is expressed early during viral replication and is thought to play an important role in the persistence of infection (Kim et al., J. Virol., 63:3708-3713, 1989; Klotman et al., Proc. Nat'l Acad. Sci. U.S.A., 85:5011-5015, 1991; Kestler et al., Cell, 65:651-662, 1991; Harris, J. GenVirol., 77:2379-2392, 1996).

Prior to the present invention, the best documents biological activity of Nef was the down-regulation of immunologically relevant surface proteins such as CD4 and MHCI (Oldridge et al., Trends Cell. Biol., 8:302-305, 1998). Numerous studies culminated in the conclusion that Nef acts as a modulator of intracellular activation pathways (see discussion in Foti et al., J. Biol. Chem., 274(49):34765-34772, 1999). In particular, Nef has been shown to interact with serine/threonine protein kinases (Biggs et al., J. Mol. Biol. 290:21-35, 1999; Saksela et al., EMBO. J. 14:484-491, 1995; Lee et al., Cell, 85:931-942, 1996; Lee et al., EMBO J. 14:5006-5015, 1995; Collette et al., J. Biol. Chem., 271:633-6341, 1996; Bauer et al., Immunity, 6:283-291, 1997).

The protein crystal structure of Nef has been resolved and it shows that Nef possesses a core region having a proline-rich motif, which binds with high affinity to the SH3 domains of Src family protein-tyrosine kinases (Arold et al., Structure, 5(10): 1361-1372, 1997; Arold et al., TIBS 26(6):356-3363, 2001). The Src family of non-receptor tyrosine kinases consists of nine members: Src, Lck, Hck, Fyn, Fgr, Yes, Blk, Lyn and Yrk (Tsygankov et al., Stem Cells (Dayton) 11:371-380, 1993). The affinity of Nef for Hck is the highest known for any SH3-mediated protein binding activity (Lee et al., EMBO J., 14:5006-5015). The Src family kinases are involved in numerous signaling events including cell growth (Willman et al., Blood, 77:726-734, 1991), T cell antigen receptor signaling (Mustelin, Immunity, 1:351-356, 1994), phagocytosis (Welch et al., J. Biol. Chem., 272:102-109, 1997), Fc receptor signaling (Durden et al., J. Immunol., 154:4039-4047, 1995; Wang et al., J. Exp. Med., 180:1165-1170, 1994), integrin signaling (Lowell et al., J. Cell Biol., 133:859-910, 1996), apoptosis (Di Somma et al., FEBBS Lett., 363:101-104, 1995) and calcium signaling (Foti et al., J. Biol. Chem., 274(49):34765-34772, 1999). Thus, those of skill in the art have identified numerous and varied functions for Nef, however, the present invention for the first time demonstrates that expression of HIV-1 Nef in podocytes results in alterations in gene expression, which cause dedifferentiation of the podocytes and ultimately leads to HIV associated nephropathy. These deleterious effects are mediated through the SH3 binding of Nef to Src family kinases. While the specific examples of the present invention are provided with respect to HIVAN, it should be understood that HIV-1 Nef expression in other non-lymphoid tissues leads to other secondary diseases associated with HIV infection.

Treatment of HIVAN and such other secondary diseases that result from the expression of HIV-1 Nef in non-lymphoid tissues can be achieved through the inhibition, ablation, depletion or other reduction of the Nef/Src interaction. As such, the present invention contemplates the use of small molecules, peptides, peptidomimetic, antibodies, nucleic acids to disrupt the interaction of Nef/Src and thereby effect a beneficial outcome. Methods and compositions for achieving such a beneficial outcome are described in greater detail herein below.

B. Nef Causes Hyperproliferation of Podocytes in Nephropathy

The present invention demonstrates the involvement of Nef in the dedifferentiation and proliferation of podocytes which ultimately results in HIVAN. The present section briefly describes the role of podocytes in HIVAN.

The anatomy of the kidney and the architecture of its cells are closely related to its function as the organ chiefly responsible for the elimination of waste and regulation of the blood's chemical balance. The blood filtration apparatus of the kidney is the glomerulus, whose major filtration surface consists of a basement membrane covered by fenestrated endothelial cells and specialized epithelial cells, called podocytes. Podocytes have delicate interdigitating foot processes that cover the exterior basement membrane surface of the glomerular capillary. Podocytes are responsible in part for the charge and size filtration characteristics of the glomerulus. When a dysfunction of glomerular filtration occurs, proteins from the blood can leak into the urine and illness and death can result. The major anatomic abnormality associated with this dysfunction is dedifferentiation of the podocyte foot processes.

The cell cycle is ultimately controlled by a balance between positive (cyclins) and negative (cyclin-dependent kinase inhibitors) cell cycle regulatory proteins. Cyclin E plays a pivotal role in G1/S transition, whereas cyclin A promotes the S/G2/M transition. Cyclin-dependent kinase inhibitors p21, p27 and p57 inhibit cyclin E in G1 and cyclin A in S phase (Shankland, et al., Am. J. Physiol. Renal Physiol. 278:F515-F529, 2000).

Podocytes are terminally differentiated cells, exhibiting a quiescent phenotype. Normal mature podocytes express p27 and p57, and the loss of p27 and p57 is seen in collapsing glomerulopathy (Barisoni et al., Kidney Int. 58:137-143, 2000; Shankland, et al., Am. J. Physiol. Renal Physiol. 278:F515-F529, 2000). In contrast, normal mature podocytes do not express cyclin A and Ki-67, a cell proliferation marker, whereas the expression of cyclin A and Ki-67 is detected in podocytes overlaying areas of glomerular collapse, indicating a re-entry of the cell cycle and proliferation (Barisoni et al. Kidney Int. 58:137-143, 2000). The loss of p27 and p57 leading to expression of cyclin A has been suggested to account for the activation of podocyte proliferation of collapsing glomerulopathy (Barisoni et al., Kidney Int. 58:137-143, 2000). The present invention for the first time shows that Nef downregulates the expression of p27 and p57, and upregulates the expression of cyclin A, cyclin E, and Ki-67, thereby showing that Nef causes the epithelial hyperplasia in collapsing glomerulopathy.

C. Methods of Treating Disorders Associated with HIV Infection

The inventors have shown that the expression of HIV-1 Nef in non-lymphoid cells infected with HIV-1 results in the expression of genes that are deleterious to the normal phenotype. In an exemplary embodiment, it is shown herein that the nephropathy seen in HIV infection is caused by an expression of HIV-1 Nef in the podocytes. The Nef, through an interaction with the SH3 binding domain of Src family tyrosine kinases, induces dedifferentiation of the podocytes. This dedifferentiation mimics the dedifferentiation of podocytes seen in HIV-infected subjects. Taking these findings into account, the present invention is directed to methods and compositions for the treatment of disorders that appear in HIV infection. These methods and compositions are designed to disrupt or interfere with the interaction of Nef with Src family tyrosine kinases. The compositions may be any composition that interferes with, and reduces, inhibits or decreases the Nef/Src tyrosine kinase interaction. As such, the present invention specifically contemplates peptides, small molecule inhibitors, anti-Nef antibodies, peptidomimetics, antisense nef nucleic acids, and the like.

Although there have been reports that the genes of HIV-1 are expressed in non-lymphoid tissues and that a transgenic mouse which expresses an HIV-1 plasmid construct deleted for gag and pol (i.e., that expresses the env, tat, rev, vif, vpr, vpu and nef genes of HIV-1; Dickie et al., Virology 185:109-119, 1991; Kopp et al., Contrib Nephrol 107:194-204, 1994) exhibits pathological characteristics of HIVAN, the present invention provides the first evidence that Nef is the central mediator of the nephropathic response. Essentially, Nef is found to activate members of the Src tyrosine kinase family through binding with the SH3 domain of these kinases. HIV-1 Nef expression modulates the expression of a number of genes involved in the nephropathic response (e.g., there is an up-regulation of one or more of the genes selected from the group consisting of PCNA, clusterin and b-raf and a down regulation of one or more of the genes selected from the group consisting of p57, ezrin, Hepatocyte Nuclear Factor 3, pur alpha, CTCF, c-erb and transducer of Erb-2, see e.g., Examples 4 through 6 herein below).

Thus, in a particular embodiment of the present invention, there are provided methods for the treatment of a disease associated with HIV-1 infection, or nephropathy in general or HIVAN in particular. These methods exploit the inventors' observation, described in detail below, that Nef regulates the expression of genes involved in the nephropathic response through its interaction with Src family tyrosine kinases. At its most basic, this embodiment will function by reducing the in vivo activity of Nef in individuals infected with HIV-1. This may be accomplished by one of several different mechanisms. First, one may block the expression of the Nef protein. Second, one may directly block the function of the Nef protein by providing an agent that binds to and/or inactivates the Nef protein. And third, one may indirectly block the effect of Nef by interfering with one or more targets of Nef, such as one or more of the Src family tyrosine kinases, or a gene influenced by the Nef/Src tyrosine kinase activity, such as for example, one or more of the genes discussed in Examples 4 through 6 below.

The therapeutic compositions of the present invention may be administered in a manner similar to the administration of current treatments for kidney disease, such as regional deliver to the kidney cells, dialysis, and the like. For a detailed review of methods of treating nephropathy, those of skill in the art are referred to Winston, et al. 2000 (Semin Nephrol. 20(3):293-8), which provides an overview of the regimens currently being used for treating nephropathy. Such treatments include highly active antiretroviral therapy (HAART; Wali et al., Lancet, 352:783-784, 1998), steroids (Appel et al., Ann Intern Med., 113:892-893, 1990; Briggs et al., Am. J. Kidney Dis., 28:618-621, 1996; Watterson et al., Am. J. Kidney Dis., 29 624-626, 1997; Smith et al., Am J. Med. 97: 145-151, 1994; Smith et al., Am J. Med., 101:41-48, 1996) and use of converting enzyme inhibitors (Ruggenenti et al., Lancet, 354:359-364, 1999; Lewis et al., New Engl. J. Med., 329:1456-1562, 1993; Burns et al., J. Am Soc. Nephrol., 8:1140-1146, 1997). It is contemplated that any such techniques may be used in conjunction with the present invention to achieve a therapeutic intervention in HIVAN using the methods and compositions of the present invention. The therapeutic formulations can also be for oral administration in a tablet form to be swallowed or to be dissolved under the tongue. These medicaments can also be provided as a patch to be worn on the skin, or as a topical cream to be applied to the skin.

Any of the therapeutic compositions of described herein below can be used either alone or in combination with each other. Further, the present invention also contemplates the use of the following compositions in combination with standard treatments presently being used for the treatment of HIV related disorders. It is specifically contemplated that the therapeutic compositions of the present invention may, for example be employed as part of the antiviral cocktail presently being used for the management of HIV infection.

a. Peptide Compositions

The present invention provides peptides that may be used as inhibitors of the interaction of Nef with Src family tyrosine kinases. Exemplary peptides include:

TABLE 1

| | | |
|---|---|---|
| Name | Residues | Peptide |

| | | |
|---|---|---|
| Tat/wt-Nef | 29 | Biotinyl-βAla-YARAAARQARAVGFPTPQVPLRPMTY |
| | | (SEQ ID NO:1) |
| Tat/Nef^{L76A} | 29 | Biotinyl-βAla-YARAAARQARAVGFPVTPQVPARPMTY |
| | | (SEQ ID NO:2) |
| Pen/wt-Nef | 34 | Biotinyl-βAla-RQIKIWFQNRRMKWKKVGFPVTPQVPLRPMTY |
| | | (SEQ ID NO:3) |
| Pen/wt-Nef^{L76A} | 34 | Biotinyl-βAla-RQIKIWFQNRRMKWKKVGFPVTPQVPARPMTY |
| | | (SEQ ID NO:4) |
| Pen*/wt-Nef | 25 | Biotinyl-βAla-RRMKWKKVGFPVTPQVPLRPMTY |
| | | (SEQ ID NO:5) |
| Pen*/Nef^{L76A} | | Biotinyl-βAla-RRMKWKKVGFPVTPQVPARPMTY |
| | | (SEQ ID NO:6) |

These peptides were designed to contain the PXXP motif of Nef and therefore may serve as competitive inhibitors of the wild-type Nef expressed in HIV infected cells.

In discussing the sequences of the peptides of the invention, the present application employs the conventional abbreviations for the amino acids as follows:

Alanine, Ala, A; Arginine, Arg, R; Asparagine, Asn, N; Aspartic acid, Asp, D; Cysteine, Cys, C; Glutamine, Gln, Q; Glutamic Acid, Glu, E; Glycine, Gly, G; Histidine, His, H; Isoleucine, Ile, I; Leucine, Leu, L; Lysine, Lys, K; Methionine, Met, M; Phenylalanine, Phe, F; Proline, Pro, P; Serine, Ser, S; Threonine, Thr, T; Tryptophan, Trp, W; Tyrosine, Tyr, Y; Valine, Val, V; Aspartic acid or Asparagine, Asx, B; Glutamic acid or Glutamine, Glx, Z; Norleucine, Nle; Acetyl-glycine (Ac)G; Any amino acid, Xaa, X. Additional modified amino acids known to those of skill in the art also may be used.

Table 1A: The following table lists the consensus sequence for HIV-1 nef (primary sequence) and provides exemplary variations of the sequence that may be useful in the present invention.

| | |
|---|---|
| primary sequence: | VGFPVRPQVPLRPMTY |
| variable amino acids: | I VSAT KL AISR |
| Y K RT ELAH | |
| R A NΦ T F | |
| A M G S | |
| W C P | |
| S Q I | |
| H Q | |
| Q | |
| C | |

In the above table, the amino acids of the primary sequence is comprised of 16 amino acids. In the following description, the residues at each of the positions is referred to using a “P” followed by a number. For example, the primary sequence is P¹P²P³P⁴P⁵P⁶P⁷P⁸P⁹P¹⁰P¹²P³P¹⁴P¹⁵P¹⁶ in which P¹ is G, P³ is F, P⁴ is P, P⁵ is V, P⁶ is R, P⁷ is P, P⁸ is Q, P⁹ is V, P¹⁰ is P, P¹¹ is L, P¹² is R, P¹³ is P, P¹⁴ is M, P¹⁵ is T, and P¹⁶ is Y. The peptides may be designed to comprise a sequence that increases the uptake of the peptides. For example, the peptides may comprise an N-terminal sequence that enhances the permeability of the molecule. In the peptides of the present invention that are contemplated to be useful as inhibitors of the Nef/Src interaction, peptides which comprise V or I in P¹ are particularly preferred; at P², the amino acid residue is preferably G; the amino acid residue at P³ may preferably be F or it also may be V; P⁴ may comprise P, S, Y, R or A; at P⁵ the residue is preferably V but also may be A; P⁶ is preferably R but also may be T, K, A, M, W, S, H, Q, or C; P⁷ required to be P; P⁸ is preferably Q also may be K, R, N, G, C, or Q; P⁹ in preferred peptides is V, but also may be T, L or any non-polar residue; P¹⁰ is required to be P required; P¹¹ in preferred peptides is L; in preferred P¹² is R; P¹³ is preferably P but also may be A, E, T, S, P, I or Q; M in P¹⁴ is preferred but P¹⁴ also may be I or L; T in P15 is preferred but P¹⁵ also may be S or A; Y in P¹⁶ is preferred but P¹⁶ also may be R, H or F. One of skill in the art will be able to construct numerous variations of peptides for use in the present invention using these preferred amino acids at the indicated positions.

Exemplary peptides derived from the above teachings include but are not limited to IGVSATPKLPLRAISR (SEQ ID NO:7); VGFYVKPRTPLRELAH (SEQ ID NO:8); VGFRVAPNNPLRTMTF (SEQ ID NO:9); VGFAVMPGVPLRSMTY (SEQ ID NO:10); VGFPVWPCVPLRPMTY (SEQ ID NO:11); VGFPVSPQVPLRIMTY (SEQ ID NO:12); VGFPVHPQVPLRQMTY (SEQ ID NO:13); VGFPVQPQVPLRQMTY (SEQ ID NO:14); VGFPVCPQVPLRQMTY (SEQ ID NO:15). As used herein in the peptides, the symbol “φ” refers to all non-polar amino acid residues.

The peptides of the present invention may be tested for their effect in kinase assays. In an exemplary assay, the cells, for example podocyte cells, are cultured in the presence and absence of the cell permeable versions of the peptides of the present invention. Following lysis, the cells are incubated with anti-Hck antibody (Santa Cruz Biotechnology, Calif.) and protein A beads, or with anti-Src antibody (Parsons, et al., J. Virol. 59:755-758) or other Src family kinase antibody and anti-mouse antibody prebound to protein A/G beads. The immunocomplex formed is washed and resuspended in kinase buffer, and incubated with 25 μCi of γ-³²P ATP. After 20 min of incubation at 32° C., the reaction is stopped by addition of protein loading dye. This mixture is boiled and electrophoresed through an SDS-10% polyacrylamide gel, transferred to Immobilon-P membrane (Millipore Corp., Bedford, Mass.), followed by autoradiography to determine the specific activity of the Src family tyrosine kinase antibody. A comparison of the specific activity of the kinase in the presence and absence of the peptides will allow a facile determination of the inhibitory effects of the peptides.

The peptide inhibitors of the present invention may be any length of amino acids so long as the amino acids are of a sufficient length to interfere with the interaction of Nef with a Src family tyrosine kinase (referred to herein as Nef/Src interaction). Preferably, the novel peptide inhibitors of the Nef/Src interaction are at least about five amino acids in length, in certain embodiments the novel peptides of the present invention may comprise a contiguous amino acid sequence of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more amino acids.

In considering the particular amino acid to be positioned at any of the positions of the peptide inhibitors, it may be useful to consider the hydropathic index of amino acids at each of the positions in a peptide known to be-an effective inhibitor of the Nef binding to the SH3 domain of a Src family tyrosine kinase, and substitute a given amino acid with one of a similar hydropathic index. It is accepted that the relative hydropathic character of the amino acid contributes to the secondary structure of a resultant protein or peptide, which in turn defines the interaction of that protein with other molecules, for example, enzymes, substrates, receptors, DNA, antibodies, antigens, and the like. Each amino acid has been assigned a hydropathic index on the basis of their hydrophobicity and charge characteristics (Kyte & Doolittle, J. Mol. Biol., 157(1):105-132, 1982, incorporated herein by reference). Generally, amino acids may be substituted by other amino acids that have a similar hydropathic index or score and still result in a protein with similar biological activity i.e., still obtain a biological functionally equivalent protein or peptide. In the context of the peptides of the present invention, a biologically functionally equivalent protein or peptide will be one which still retains its ability to be an antagonist of the Nef binding to a SH3 domain of a Src family tyrosine kinase.

In addition, the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As such, an amino acid can be substituted for another having a similar hydrophilicity value and still obtain a biologically equivalent and immunologically equivalent protein.

In addition to the novel peptide inhibitors described above, the present invention further contemplates the generation terminal additions, also called fusion proteins or fusion polypeptides, of the peptides described above or identified according to the present invention. This fusion polypeptide generally has all or a substantial portion of the native molecule (i.e., the peptide inhibitors discussed above), linked at the N- and/or C-terminus, to all or a portion of a second or third polypeptide. It is contemplated that the fusion polypeptide may be produced by recombinant protein production or by automated peptide synthesis.

In preferred aspects of the invention, peptides were synthesized according to methods known to those of skill in the art (Carter, et al., Biotechnology, 10(5): p. 509-13, 1992; Chen, et al., in Peptides: Wave of the Future—Proceedings of the 17th Arnerican Peptide Symposium, ed. M. Lebl and R. Houghten, Editors., Mayflower Scientific Ltd.: Kingswinford, UK. pp 206-207 and pp 318-319, 2001). In such embodiments, the peptide ligands were synthesized using Mimotopes (Clayton, Australia) SynPhase™ acrylic-grafted polypropylene solid support, in 96 well microtiter plates with Fmoc chemistry. The Fmoc-protected amino acids and reagents used were from AnaSpec (San Jose, Calif.), Chemlmpex (Wood Dale, Ill.), and NovaBiochem (San Diego, Calif.). The couplings were carried out in the DMF solution of HOBt/HBTU/DIEA. The washing steps were performed in a bath of MeOH or DMF. Reagent R was used to cleave the peptides from solid support upon completion. The crude peptide products were analyzed and characterized via high throughput LC-MS (a Shimadzu VP series HPLC system and a PE Sciex API 165 mass spectrometer). The peptides were purified by Gilson (Middleton, Wis.) HPLC systems with 215 liquid handlers when their purity fell below 85%.

General principles for designing and making fusion proteins are well known to those of skill in the art. For example, fusions typically employ leader sequences from other species to permit the recombinant expression of a protein or peptide in a heterologous host. Another useful fusion includes the addition of an immunologically active domain, such as an antibody epitope, to facilitate purification of the fusion polypeptide. Inclusion of a cleavage site at or near the fusion junction will facilitate removal of the extraneous polypeptide after purification. The recombinant production of these fusions is described in further detail elsewhere in the specification. Other useful fusions include linking of functional domains, such as active sites from enzymes, glycosylation domains, cellular targeting signals or transmembrane regions.

There are various commercially available fusion protein expression systems that may be used to provide a tagged sequence in this context of the present invention. Particularly useful systems include but are not limited to the glutathione S-transferase (GST) system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.). These systems are capable of producing recombinant polypeptides bearing only a small number of additional amino acids, which are unlikely to affect the biologically relevant activity of the recombinant fusion protein. For example, both the FLAG system and the 6×His system add only short sequences, both of which are known to be poorly antigenic and which do not adversely affect folding of the polypeptide to its native conformation. Another N-terminal fusion that is contemplated to be useful is the fusion of a Met-Lys dipeptide at the N-terminal region of the protein or peptides.

In addition to creating fusion polypeptides, it is contemplated that the fusion proteins or the peptide inhibitors may be further modified to incorporate, for example, a label or other detectable moiety.

Preferred peptide inhibitors will comprise internally quenched labels that result in increased detectability after cleavage of the peptide inhibitors. The peptide inhibitors may be modified to have attached a paired fluorophore and quencher including but not limited to 7-amino-4-methyl coumarin and dinitrophenol, respectively. Other paired fluorophores and quenchers include bodipy-tetramethylrhodamine and QSY-5 (Molecular Probes, Inc.). In a variant of this assay, biotin or another suitable tag may be placed on one end of the peptide to anchor the peptide to a substrate assay plate and a fluorophore may be placed at the other end of the peptide. Useful fluorophores include those listed above as well as Europium labels such as W8044 (EG&g Wallac, Inc.).

Further, the peptides may be labeled using labels well known to those of skill in the art, e.g., biotin labels are particularly contemplated. The use of such labels is well known to those of skill in the art and is described in, e.g., U.S. Pat. No. 3,817,837; U.S. Pat. No. 3,850,752; U.S. Pat. No. 3,996,345 and U.S. Pat. No. 4,277,437. Other labels that will be useful include but are not limited to radioactive labels, fluorescent labels and chemiluminescent labels. U.S. patents concerning use of such labels include for example U.S. Pat. No. 3,817,837; U.S. Pat. No. 3,850,752; U.S. Pat. No. 3,939,350 and U.S. Pat. No. 3,996,345. Any of the peptides of the present invention may comprise, one two or more of any of these labels.

b. Nucleic Acid Based Perturbation of Nef/Src Interaction

The Nef/Src interaction may be disrupted through the use of nucleic acid based techniques to block the expression of Nef, and therefore, to perturb the Nef/Src binding reaction. Polynucleotide gene products which are useful in this endeavor include antisense polynucleotides, ribozymes, RNAi, and triple helix polynucleotides that modulate the expression of Nef. Antisense polynucleotides and ribozymes are well known to those of skill in the art. Crooke and B. Lebleu, eds. Antisense Research and Applications (1993) CRC Press; and Antisense RNA and DNA (1988) D. A. Melton, Ed. Cold Spring Harbor Laboratory Cold Spring Harbor, N.Y. Anti-sense RNA and DNA molecules act to directly block the translation of mRNA by binding to targeted mRNA and preventing protein translation. An example of an antisense polynucleotide is an oligodeoxyribonucleotide derived from the translation initiation site, e.g., between −10 and +10 regions of the relevant nucleotide sequence.

As indicated above, the DNA and protein sequences for HIV Nef are published and disclosed in e.g., U.S. Pat. No. 5,705,612; U.S. Pat. No. 6,261,564; U.S. Pat. No. 5,968,514; Genbank Accession Nos. CAC 38428; CAC 38437; NP 588661; NP 057857. Those of skill in the art are referred to the Genbank Database at www.ncbi.nlm.nih.gov, which lists HIV-1 Nef sequences known to those of skill in the art and also lists related Nef sequences from Simian Immunodeficiency Virus, which may be useful in certain aspects of the present invention. Although antisense sequences may be full length genomic or cDNA copies, they also may be shorter fragments or oligonucleotides e.g., polynucleotides of 100 or less bases. Although shorter oligomers (8-20) are easier to make and more easily permeable in vivo, other factors also are involved in determining the specificity of base pairing. For example, the binding affinity and sequence specificity of an oligonucleotide to its complementary target increases with increasing length. It is contemplated that oligonucleotides of 8, 9, 10, 11, 12, 13, 14, 15, 16, 17, 18, 19, 20, 25, 30, 35, 40, 45, 50, or more base pairs will be used.

Ribozymes are enzymatic RNA molecules capable of catalyzing the specific cleavage of RNA. The mechanism of ribozyme action involves sequence specific interaction of the ribozyme molecule to complementary target RNA, followed by an endonucleolytic cleavage. Within the scope of the invention are engineered hammerhead or other motif ribozyme molecules that specifically and efficiently catalyze endonucleolytic cleavage of RNA sequences encoding protein complex components.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate targets may also be evaluated by testing their accessibility to hybridization with complementary oligonucleotides, using ribonuclease protection assays. See, Draper PCT WO 93/23569; and U.S. Pat. No. 5,093,246.

Nucleic acid molecules used in triple helix formation for the inhibition of transcription are generally single stranded and composed of deoxyribonucleotides. The base composition must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Another technique that is of note for reducing the or disruption the expression of a gene is RNA interference (RNAi). The term “RNA interference” was first used by researchers studying C. elegans and describes a technique by which post-transcriptional gene silencing (PTGS) is induced by the direct introduction of double stranded RNA (dsRNA: a mixture of both sense and antisense strands). Injection of dsRNA into C. elegans resulted in much more efficient silencing than injection of either the sense or the antisense strands alone (Fire et al., Nature 391:806-811,1998). Just a few molecules of dsRNA per cell is sufficient to completely silence the expression of the homologous gene. Furthermore, injection of dsRNA caused gene silencing in the first generation offspring of the C elegans indicating that the gene silencing is inheritable (Fire et al., Nature 391:806-811, 1998). Current models of PTGS indicate that short stretches of interfering dsRNAs (21-23 nucleotides; siRNA also known as “guide RNAs”) mediate PTGS siRNAs are apparently produced by cleavage of dsRNA introduced directly or via a transgene or virus. These siRNAs may be amplified by an RNA-dependent RNA polymerase (RdRP) and are incorporated into the RNA-induced silencing complex (RISC), guiding the complex to the homologous endogenous mRNA, where the complex cleaves the transcript.

While most of the initial studies were performed in C elegans, RNAi is gaining increasing recognition as a technique that may be used in mammalian cell. It is contemplated that RNAi may be used to disrupt the expression of a gene in a tissue-specific manner. By placing a gene fragment encoding the desired dsRNA behind an inducible or tissue-specific promoter, it should be possible to inactivate genes at a particular location within an organism or during a particular stage of development. Recently, RNAi has been used to elicit gene-specific silencing in cultured mammalian cells using 21-nucleotide siRNA duplexes (Elbashir et al., Nature, 411:494-498, 2001). In the same cultured cell systems, transfection of longer stretches of dsRNA yielded considerable nonspecific silencing. Thus, RNAi has been demonstrated to be a feasible technique for use in mammalian cells and could be used for assessing gene function in cultured cells and mammalian systems, as well as for development of gene-specific therapeutics.

Anti-sense RNA and DNA molecules, ribozymes, RNAi and triple helix molecules can be prepared by any method known in the art for the synthesis of DNA and RNA molecules. These include techniques for chemically synthesizing oligodeoxyribonucleotides well known in the art including, but not limited to, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably or transiently into cells.

c. Blocking Nef Function

In another embodiment it may be desirable to block the function of the Nef polypeptide rather than its expression. This can be accomplished through the use of an organochemical composition (i.e., a small molecule inhibitor) that interferes with the function of the Nef, by use of an antibody that blocks an active site or binding site on the Nef polypeptide (i.e., the SH3 binding site of a Nef polypeptide), or by use of a molecule that mimics the Nef target (i.e., the SH3 domain of Src family tyrosine kinases).

With respect to small molecule inhibitors such compounds may be identified through standard screening assays. For example, it is known that Nef interacts with Src family tyrosine kinases through binding to the SH3 domain of such kinases. Various candidate substances can be contacted with Nef followed by further determination of the ability of treated Nef to bind to an SH3 domain of a Src family tyrosine kinase. An agent that inhibits such binding will be a useful for blocking the Nef/Src interaction. Small molecule inhibitors of Nef expression that inhibit LTR-mediated transcription or splicing or Nef-specific mRNAs are contemplated to be useful in the present invention. For the former example, flavopiridol ameliorates HIVAN in transgenic mice by inhibiting cellular Cdk9, an enzyme required for HIV-1 Tat to induce LTR-mediated virus transcription. Flavopiridol, derivatives thereof and compounds in the same class as flavopiridol may be used in the present invention.

The methods by which antibodies are generated are well known to those of skill in the art. Antibodies that bind to Nef maybe screening for other functional attributes, e.g., effect on tyrosine kinase specific activity, effect on expression of genes regulated by Nef (see Table 3 in the Examples for exemplary genes that are regulated by Nef).

A particularly useful antibody for blocking the action of Nef is a single chain antibody. Methods for the production of single-chain antibodies are well known to those of skill in the art. The skilled artisan is referred to U.S. Pat. No. 5,359,046, (incorporated herein by reference) for such methods. A single chain antibody, preferred for the present invention, is created by fusing together the variable domains of the heavy and light chains-using a short peptide linker, thereby reconstituting an antigen binding site on a single molecule.

Single-chain antibody variable fragments (Fvs) in which the C-terminus of one variable domain is tethered to the N-terminus of the other via a 15 to 25 amino acid peptide or linker, have been developed without significantly disrupting antigen binding or specificity of the binding. These Fvs lack the constant regions (Fc) present in the heavy and light chains of the native antibody.

With respect to inhibitors that mimic Nef targets, the use of mimetics provides one example of custom designed molecules. Such molecules may be small molecule inhibitors that specifically inhibit Nef protein activity or binding to a Src family tyrosine kinase. Such molecules may be sterically similai to the actual target compounds, at least in key portions of the target's structure and or organochemical in structure. Alternatively these inhibitors may be peptidyl compounds, these are called peptidomimetics. Peptide mimetics are peptide-containing molecules which mimic elements of protein secondary structure. The underlying rationale behind the use of peptide mimetics is that the peptide backbone of proteins exists chiefly to orient amino acid side chains in such a way as to facilitate molecular interactions, such as those of ligand and receptor. An exemplary peptide mimetic of the present invention would, when administered to a subject, bind Nef in a manner analogous to the Src family tyrosine kinase SH3 domain binding to wild-type Nef.

Successful applications of the peptide mimetic concept have thus far focused on mimetics of β-turns within proteins, which are known to be highly antigenic. Likely β-turn structures within an antigen of the invention can be predicted by computer-based algorithms as discussed above. Once the component amino acids of the turn are determined, mimetics can be constructed to achieve a similar spatial orientation of the essential elements of the amino acid side chains.

d. Blocking of a Nef Target

As discussed above, one of the benefits of the present invention is the identification of targets upon which Nef acts. These targets may be binding partners such as members of the Src tyrosine kinase family, or other genes that are upregulated by an activated Nef interaction with an Src SH3 domain of a Src family tyrosine kinase. In order to prevent Nef from interacting with these targets, one may take a variety of different approaches. For example, one may generate antibodies against the target and then provide the antibodies to the subject in question, thereby blocking access of Nef to the target molecule.

In yet another embodiment, antisense methodologies may be employed in order to inhibit expression of the Nef-induced target gene. Alternatively, one may design a polypeptide or peptide mimetic that is capable of interacting with the Nef target in the same fashion as Nef, but without any Nef-like effect on the target.

In a preferred embodiment, the present invention will provide an agent that binds competitively to Src family tyrosine kinase SH domain. In a more preferred embodiment, the agent will have an even greater affinity for the Src family tyrosine kinase SH3 domain than Nef does. Affinity for the Src family tyrosine kinase SH3 domain can be determined in vitro by performing kinetic studies on binding rates.

Other compounds may be developed based on computer modeling and predicted higher order structure, both of the Nef molecule and of the identified target molecules. This approach has proved successful in developing inhibitors for a number of receptor/ligand interactions. To this effect, the crystal structure of wild-type Nef is known to those of skill in the art (Arold et al., Structure, 5(10): 1361-1372, 1997; Arold et al., TIBS 26(6):356-3363, 2001).

The crystal structure of HIV-1 Nef in complex with a Src family SH3 domain (Lee et al., Cell, 85, 931-942 1996) shows that the complex is formed not only by the classical SH3 domain recognition of a conserved PXXP sequence on Nef, but also stabilized by a complementary interaction of a loop of the SH3 domain (the RT loop) with a surface-exposed binding pocket on Nef. The latter interaction is particularly important and confers the high-affinity and specificity for this SH3 domain/protein complex. Specifically, in the Nef-SH3 domain complex, the unique RT loop of the SH3 domain (between the first and second strands of the protein) extends over the surface of Nef and intercalates the side-chain of Ile-96 of SH3 into a pocket formed between helices aA and aB of Nef. This solvent-accessible crevice is highly hydrophobic, in which the bulky hydrophobic isoleucine side chain at position 96 of the SH3 domain packs against the conserved side chains of Leu-87, Phe-90, Trp-113 and Ile-114 of Nef.

The unique features of the RT-loop binding between the two proteins make this interaction an ideal target site for designing small molecular chemical inhibitors that bind selectively to this cavity in order to disrupt the interaction of Nef with the Src SH3 domain. It would be desirable to select this specific target because: (i) targeting the viral Nef protein rather than the human SH3 domain protein to block the Nef/SH3 association is preferable for therapeutic agent development, because targeting the viral protein can greatly minimize potential side effects or toxicity of drug molecules that result from their possible interferences on biochemical or biological functions of mammalian protein (such as, for example the Src-like tyrosine kinase proteins); (ii) the surface-exposed RT loop-binding site on Nef makes it readily accessible for interactions with designed chemical ligands; and (iii) the protein-protein interactions are largely hydrophobic in nature, which makes it easier to identify small molecular chemicals that bind specifically to the site. These initial binding chemical compounds can help chemical lead optimization to improve ligand binding-affinity and selectivity.

The detailed structural information of the complex can guide one to build specific small chemical entities that interact with this site. One such a structure-based approach is to use nuclear magnetic resonance (NMR) spectroscopy and a linked-fragment approach to identify chemical leads for development of specific ligands for therapeutic targets (Hajduk et al., Science, 278, 498-499, 1997; Moore, Curr Opin in Biotech., 10, 54-58, 1999; Pellecchia et al., Nature Reviews Drug Discovery, 1, 211-219, 2002). The novel ligands are chemical entities constructed from building blocks identified from NMR-based screening and optimized for binding to a target protein.

The NMR-based chemical compound screening has significant advantages that make it preferable even over the newest methods of high-throughout screening of natural products or combinatorial chemical libraries (Fejzo et al., Chem. Biol. 6, 755-769, 1999; Hajduk et al., J Am Chem Soc 119, 5818-5827, 1997; Hajduk et al., Bioorganic & Medicinal Chemistry Letters 9, 2403-2406, 1999; Pellecchia et al., J Biomol NMR 22, 165-173, 2002). These unique advantages include (i) structure-based and selective screening for specific sites on a target protein; (ii) rapid and reliable screening of weak binding ligands; (iii) a large virtual library of small chemical compounds; and (iv) independent optimization of individual chemical fragments. The resulting linked chemical compounds with high affinity and selectivity are then subject to detailed structure-based analysis of their interactions with the target protein using a combination of NMR and computational modeling techniques. Refinement, chemical diversification through various chemical linkages and selectivity enhancement are achieved at this stage.

For a detailed description of methods for identification of small molecule inhibitors those of skill in the art are referred to WO01/51521, which describes the three-dimensional structure of a complex between phosphotyrosine binding domain of Suc 1-associated neurotrophic factor target protein and the SNT binding site of fibroblast growth factor receptor. Rational drug design predicated on the three-dimensional structure of this interaction is described in detail. It is contemplated that the techniques therein may be used for rational drug design to identify agents that can inhibit the deleterious effects of Nef binding to Src-like tyrosine kinases. For example such a method would involve identifying a compound that destabilizes the Nef/Src interaction and would involve obtaining a set of atomic coordinates that define the three dimensional structure of a Nef/Src interaction. These coordinates are determined using a complex which comprises an HIV Nef protein interacting with a Src-like tyrosine kinase (Nef/Src complex). The next step involves performing rational drug design with the atomic coordinates to select a drug that interferes with the Nef/Src complex at a given site (e.g., at the RT loop). This rational drug design is preferably performed in conjunction with computer modeling. Upon selection of the candidate drug, the candidate is contacted with a Nef/Src complex comprising a full length or fragment of Nef protein and a full length or fragment of a Src-like tyrosine kinase protein. The stability of the Nef/Src complex is monitored in the presence and absence of the candidate substance to identify a potential therapeutic agent which destabilizes the complex. Similar methods may be performed to identify a compound which inhibits the formation of the complex. Such methods are described in detail in WO01/51521.

D. Methods of Making Recombinant Cells and Transgenic Animals

As noted above, a particular embodiment of the present invention provides transgenic animals which contain an active Nef These animals exhibit all the characteristics associated with the pathophysiology of nephropathy. Transgenic animals expressing nef transgenes, recombinant cell lines derived from such animals and transgenic embryos may be useful in methods for screening for and identifying agents that repress function of Nef and thereby alleviate diseases associated with HIV-1 infection, such as, for example, HIVAN.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; which is incorporated herein by reference), Palmiter and Brinster Cell, 41(2):343-5, 1985; which is incorporated herein by reference in its entirety) and in “Manipulating the Mouse Embryo; A Laboratory Manual” 2^{nd} edition (eds. Hogan, Beddington, Costantimi and Long, Cold Spring Harbor Laboratory Press, 1994; which is incorporated herein by reference in its entirety).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which express a HIV-1 Nef polypeptide. In particularly preferred embodiments, the transgenic animals express HIV-1 Nef in non-lymphoid tissues. In still more preferred embodiments, the animals express HIV-1 Nef in kidney cells. In other preferred embodiments, the transgenic mice express HIV-1 Nef in podocytes.

In an exemplary expression construct used for the generation of the transgenic animals and recombinant cells of the present invention, a 4.125 K^{b} fragment containing the murine nephrin promoter (4145 to 8270 bp of GenBank accession number AF296764) was spliced into pcDNA3.1 (Invitrogen) using PacI/XhoI sites. The Nef gene (bp. 8787 to 9407 of Genbank accession #AF324493) was PCR amplified an then spliced downstream of the Nephrin promoter as a XhoI/EcoRV fragment. The Nephrin promoter-Nef gene fusion was liberated as a HindIII/NsiI fragment, blunted with Klenow enzyme, and then spliced into ClaI site (blunted with Klenow) of the transgenic vector, pDelboy. This vector was produced by Derrick J. Rossi (Derrick.Rossi@Helsinki.Fi) in the lab of Tomi Makela. (Rossi et al., EMBO J., 20(11):2844-56, 2001).

DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol: chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm is a UV spectrophotometer. For microinjection, DNA concentrations are adjusted to 3 μg/ml in 5 mM Tris, pH 7.4 and 0.1 mM EDTA.

Other methods for purification of DNA for microinjection are described in Hogan et al. Manipulating the Mouse Embryo (Cold Spring Harbor Laboratory, Cold Spring Harbor, N.Y., 1986), in Palmiter et al. Nature 300:611 (1982); the Qiagenologist, Application Protocols, 3^{rd} edition, published by Qiagen, Inc., Chatsworth, Calif.; and in Sambrook et al. Molecular Cloning: A Laboratory Manual (Cold Spring Harbor Laboratory, Cold Spring Harbor, N.Y., 1989).

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% CO₂ 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmaker's forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

E. Monitoring Transgene Expression

In order to determine whether the active Nef has been successful incorporated into the genome of the transgenic animal, a variety of different assays may be performed. Transgenic animals can be identified by analyzing their DNA. For this purpose, when the transgenic animal is a rodent, tail samples (1 to 2 cm) can be removed from three week old animals. DNA from these or other samples can then be prepared and analyzed by Southern blot, PCR, or slot blot to detect transgenic founder (F₀) animals and their progeny (F₁ and F₂).

a. Pathological Studies

The various F0, F1 and F2 animals that carry a transgene can be analyzed by any of a variety of techniques, including immunohistology, electron microscopy, and making determinations of total and regional hear weights, measuring podocyte cross-sectional areas and determining numbers of podocytes. Immunohistological analysis for the expression of a transgene by using an antibody of appropriate specificity can be performed using known methods. Morphometric analyses to determine regional weights, podocyte cross-sectional areas, and other factors relating to podocyte dedifferentiation can be performed using known methods. Kidneys can be analyzed for function, histology and expression of podocyte genes.

In immuno-based analyses, it may be necessary to rely on Nef-binding antibodies. A general review of antibody production techniques is provided. As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BAS). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

The immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium smegmatis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

A polyclonal antibody is prepared by immunizing an animal with an immunogen comprising a Nef polypeptide, or fragment thereof, and collecting antisera from that immunized animal. A wide range of animal species can be used for the production of antisera. Typically an animal used for production of anti-antisera is a rabbit, a mouse, a rat, a hamster, a guinea pig, a goat, a sheep, a horse, or a donkey. Because of the relatively large blood volume of rabbits, a rabbit may be a preferred choice for production of polyclonal antibodies.

To obtain monoclonal antibodies, one would also immunize an experimental animal with a Nef composition. One would then, after a period of time sufficient to allow antibody generation, obtain a population of spleen or lymph cells from the animal. The spleen or lymph cells can then be fused with cell lines, such as human or mouse myeloma strains, to produce antibody-secreting hybridomas. These hybridomas may be isolated to obtain individual clones which can then be screened for production of antibody to the desired target peptide.

It is proposed that the monoclonal antibodies of the present invention also will find useful application in standard immunochemical procedures, such as ELISA and Western blot methods, as well as other procedures which may utilize antibody specific to Nef epitopes. Additionally, it is proposed that monoclonal antibodies specific to Nef may be utilized in other useful applications. For examples, an anti-idiotype antibody to an anti-Nef antibody may well mimic a Nef binding site, thus providing a tool for the identification of Nef targets.

b. Analysis of Transgene Expression by Measuring mRNA Levels

Messenger RNA can be isolated by any method known in the art, including, but not limited to, the acid guanidinium thiocyanate-phenol: chloroform extraction method (Chomczynski and Sacchi 1987), from cell lines and tissues of transgenic animals to determine expression levels by Northern blots, RNAse and nuclease protection assays.

c. Analysis of Transgene Expression by Measuring Protein Levels

Protein levels can be measured by any means known in the art, including, but not limited to, western blot analysis, ELISA and radioimmunoassay, using one or more antibodies specific for the protein encoded by the transgene.

For Western blot analysis, protein fractions can be isolated from tissue homogenates and cell lysates and subjected to Western blot analysis as described by, for example, Harlow et al., Antibodies: A Laboratory Manual, (Cold Spring Harbor, N.Y. 1988).

For example, the protein fractions can be denatured in Laemmli sample buffer and electrophoresed on SDS-Polyacrylamide gels. The proteins are then transferred to nitrocellulose filters by electroblotting. The filters are blocked, incubated with primary antibodies, and finally reacted with enzyme conjugated secondary antibodies. Subsequent incubation with the appropriate chromogenic substrate reveals the position of the transgene-encoded proteins.

ELISAs are preferably used in conjunction with the invention. For example, an ELISA assay may be performed where Nef from a sample is immobilized onto a selected surface, preferably a surface exhibiting a protein affinity such as the wells of a polystyrene microtiter plate. The plate is washed to remove incompletely adsorbed material and the plate is coated with a non-specific protein that is known to be antigenically neutral with regard to the test antibody, such as bovine serum albumin (BSA), casein or solutions of powdered milk. This allows for blocking of nonspecific adsorption sites on the immobilizing surface and thus reduces the background caused by nonspecific binding of antisera onto the surface.

Next, the Nef antibody is added to the plate in a manner conducive to immune complex (antigen/antibody) formation. Such conditions preferably include diluting the antisera/antibody with diluents such as BSA bovine gamma globulin (BGG) and phosphate buffered saline (PBS)/Tween®. These added agents also tend to assist in the reduction of nonspecific background. The plate is then allowed to incubate for from about 2 to about 4 hr, at temperatures preferably on the order of about 25° to about 27° C. Following incubation, the plate is washed so as to remove non-immunocomplexed material. A preferred washing procedure includes washing with a solution such as PBS/Tween®, or borate buffer.

Following formation of specific immunocomplexes between the sample and antibody, and subsequent washing, the occurrence and amount of immunocomplex formation may be determined by subjecting the plate to a second antibody probe, the second antibody having specificity for the first (usually the Fe portion of the first is the target). To provide a detecting means, the second antibody will preferably have an associated enzyme that will generate a color development upon incubating with an appropriate chromogenic substrate. Thus, for example, one will desire to contact and incubate the antibody-bound surface with a urease or peroxidase-conjugated anti-human IgG for a period of time and under conditions which factor the development of immunocomplex formation (e.g. incubation for 2 hr at room temperature in a PBS-containing solution such as PBS/Tween®.

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂ in the case of peroxidase as the enzyme label. Quantitation is then achieved by measuring the degree of color generation, e.g., using a visible spectrum spectrophotometer. Variations on this assay, as well as completely different assays (radioimmunprecipitation, immunoaffinity chromatograph, Western blot) also are contemplated as part of the present invention.

Other immunoassays encompassed by the present invention include, but are not limited to those described in U.S. Pat. No. 4,367,110 (double monoclonal antibody sandwich assay) and U.S. Pat. No. 4,452,901 (Western blot). Other assays include immunoprecipitation of labeled ligands and immunocytochemistry, both in vitro and in vivo.

F. Methods of Using Recombinant Cells and Transgenic Animals

The transgenic animals of the present invention include those which have a substantially increased probability of spontaneously developing nephropathy, and in particular HIVAN, when compared with non-transgenic littermates. A “substantially increased” probability of spontaneously developing nephropathy means that, a statistically significant increase of measurable symptoms of nephropathy and kidney dysfunction is observed when comparing the transgenic animal with non-transgenic littermates.

The transgenic animals of the present invention are produced with transgenes which comprise a coding region that encodes a gene product which modulates transcription of at least one gene that is expressed in podocyte in response to a signal generated as a result of the interaction between HIV-1 Nef and a Src family tyrosine kinase.

As used herein, such a “signal” indicates any stimulus, mechanical or chemical, which results in measurable symptoms of nephropathy. Symptoms of nephropathy can be measured by various parameters including, but not limited to, left proteinuria, changes in podocyte size, proliferation and morphology, changes in podocyte gene expression and changes in kidney function. The transgenic animals of the present invention may be compared to the previously described models which express the env, tat, rev, vif vpr, vpu and nef genes of HIV-1; Dickie et al., Virology 185: 109-119, 1991; Kopp et al., Contrib Nephrol 107:1194-204, 1994) and exhibits the pathological characteristics HIVAN. Preferably, the transgenic animals of the present invention will exhibit similar or more severe manifestations of HIVAN than those described by Dickie et al., and Kopp et al. However, it should be understood that even if the Nef transgenic mice exhibit less severe manifestations than the previously described mice, the mice of the present invention better reflect HIVAN because the mice of the present invention express Nef alone, and as such express the component of the HIV-1 nef genome that is necessary and sufficient for the HIVAN phenotype.

Coding regions for use in constructing the transgenic mice include are HIV-1 Nef, however, it is contemplated that transgenic mice also may be constructed using coding regions for one or more of the other accessory proteins of HIV-1. The coding regions may encode a complete polypeptide, or a fragment thereof, as long as the desired function of the polypeptide is retained, i.e., the polypeptide can modulate transcription of at least one gene that is expressed in podocytes during nephropathy or as a result of activation of the Nef/Src family tyrosine kinase signal. The coding regions for use in constructing the transgenes of the present invention further include those containing mutations, including silent mutations, mutations resulting in a more active protein, mutations that result in a constitutively active protein, and mutations resulting in a protein with reduced activity. Inasmuch as Nef mediates the nephropathic response of an animal in response to HIV-1 infection as identified herein, the following discussion is based on an HIV-1 Nef transgenic mouse, however, it is understood that the teachings provided herein are equally applicable to other disorders in which the Nef/Src interaction is responsible for a disorder associated with HIV-1 infection. Similarly, the discussion also is applicable to other accessory protein encoding transgenes that may also affect nephropathy upstream or downstream of the effect of HIV-1 Nef.

In one embodiment of the present invention, there is provided a transgenic animal that express activated forms of HIV-1 Nef. By “activated HIV-1 nef gene,” it is meant that the HIV-1 nef gene expresses a functional protein that is capable of activating a Src family tyrosine kinase. Preferably such an activation is mediated through the binding of the Nef protein with the SH3 domain of a tyrosine kinase of the Src tyrosine kinase family. A preferred form of the animal is a mouse that contains an SH3 binding domain of wild-type HIV-1 Nef. Surprisingly, HIV-1 Nef is identified herein as responsible for the molecular and morphological changes that are characteristic of the HIV-1 nephropathic phenotype. Transgenic mice that express the env, tat, rev, vif, vpr, vpu and nef genes of HIV-1 (i.e., are deleted only for the gag/pol genes of HIV-1; Dickie et al., Virology 185:109-119, 1991; Kopp et al., Contrib Nephrol 107:194-204, 1994), exhibit the characteristics of HIVAN. For the first time, the present invention shows that out of all the possible genes of HIV-1 it is nef expression that is sufficient to induce the pathological phenotype of HIVAN.

The transgenic mice of the present invention has a variety of different uses. First, by creating an animal model in which only the HIV-1 Nef is expressed and constantly activated, the present inventors have provided a living “vessel” in which the function of HIV-1 Nef may be further dissected. For example, provision of various forms of Nef—deletion mutants, substitution mutants, insertion mutants, fragments and wild-type proteins—labeled or unlabeled, will permit numerous studies on HIVAN that were not previously possible.

In one particular scenario, the transgenic mouse may be used to elucidate the in vivo interactions and effects of Nef activation of Src family tyrosine kinases. Another use for the transgenic mouse of the present invention is the in vivo identification of a modulator of Nef activity, and ultimately of nephropathy in general and HIVAN specifically. The presence of a constitutively active HIVAN in the transgenic mouse represents a 100% Nef mediated HIV-1 induced nephropathic function. Treatment of a transgenic mouse with a putative Nef inhibitor, and comparison of the nephropathic response (e.g., synaptopodin or other gene expression, podocyte morphology, proteinuria and the like) of this treated mouse with the untreated transgenic animal, provides a means to evaluate the activity of the candidate inhibitor.

Yet another use of the Nef transgenic mouse described herein provides a new disease model for HIVAN. As shown in the data in the examples, the transgenic cells of the present invention demonstrates all the molecular and morphological features of nephropathy. A transgenic animal already exists that is being used as a model for HIVAN, but that animal expresses the entire HIV-1 genome with the exception of the gag/pol genes (Dickie et al., Virology 185:109-119, 1991; Kopp et al., Contrib Nephrol 107:194-204, 1994). Moreover, the expression of HIV-1 genes in that animal is ubiquitous. An exemplary animal of the present invention expresses far fewer genes than the gag/pol deleted mouse described by Dickie et al., (Virology 185:109-119, 1991) and Kopp et al., (Contrib Nephrol 107:194-204, 1994). In addition, another exemplary animal of the present invention expresses Nef only in kidney cells and more particularly, the animal expresses HIV-1 Nef only in the podocytes. In exemplary embodiments, expression of the Nef gene in kidney cell alone is achieved through the use of a kidney cell-specific promoter. In those embodiments where the Nef is being expressed in podocytes alone, such expression is effected through the use of a podocyte specific promoter. The nephrin promoter is one such glomerular specific promoter that may be used to target the expression of the HIV-1 Nef in the podocytes (Wong et al., Am. J. Renal. Physiol. 279:F1027-F1032, 2000). Another promoter that may be used in the present invention is beta-actin/beta-globin promoter (CX promoter) which could allow a podocyte-specific expression of a molecule of interest in kidney (Imai et al., Exp Nephrol., 7(1):63-6, 1999). In addition, the synaptopodin and podocin promoters also may be used to achieve kidney cell-specific expression. Thus, the Nef transgenic mouse provides a novel model for the study of nephropathy. This model could be exploited by treating the animal with compounds that potentially inhibit the Nef/Src interaction and treat HIV-related nephropathy. Also it is contemplated that such inhibitors may be useful in the treatment of other kidney disease as well as other disorders associated with HIV-1 infection.

G. Methods of Screening for Compositions for Treating Disorders Associated with HIV Infection

The present invention also contemplates screening of compounds for their ability to inhibit the Nef based activation of Src family tyrosine kinases. The present invention shows that this interaction is responsible for the secondary sequelae seen in HIV infection. This realization affords the ability to create cellular, organ and organismal systems which mimic these diseases, which provide an ideal setting in which to test various compounds for therapeutic activity. Particularly preferred compounds will be those useful in inhibiting HIVAN and preventing or reversing kidney disease associated with HIV infection mediated. In the screening assays of the present invention, the candidate substance may first be screened for basic biochemical activity—e.g., binding to a target molecule—and then tested for its ability to inhibit a nephropathic phenotype, at the cellular, tissue or whole.animal level.

a. Inhibitors and Assay Formats

i. Assay Formats

The present invention provides methods of screening for inhibitors of Nef activity. It is contemplated that this screening techniques will prove useful in the identification of compounds that will prevent HIV nephropathy in patients infected with HIV-1 and/or reduce such nephropathy once developed.

In these embodiment, the present invention is directed to a method for determining the ability of a candidate substance to inhibit the Nef/Src interaction, generally including the steps of:

a) providing a cell expressing HIV-1 Nef,

b) contacting said cell with a candidate modulator; and

c) monitoring said cell for change in a cellular property associated with nephropathy that occurs in the presence of said modulator.

To identify a candidate substance as being capable of inhibiting a nephropathic phenotype in the assay above, one would measure or determine various characteristics of the cell, for example, podocyte morphology, proliferation index, monitor Src family tyrosine kinases specific activity of said cell, expression of one of more nucleic acids selected from the group consisting of p57, ezrin, hepatocyte nuclear factor 3, pur alpha, CTCF, c-erb, transducer of Erb-2, PCNA, clusterin and b-raf in the presence and absence of said candidate modulator, protein excretion (where the cell is in an animal), and the like in the absence of the added candidate substance. One would then add the candidate substance to the cell and determine the response in the presence of the candidate substance. A candidate substance which modulates any of these characteristics is indicative of a candidate substance having modulatory activity. In the in vivo screening assays of the present invention, the compound is added to the cells, over period of time and in various dosages, and nephropathic response is measured.

ii. Inhibitors and Activators of Nef

An inhibitor according to the present invention may be one which exerts its inhibitory effect upstream or downstream of Nef, or on the Nef protein directly. Regardless of the type of inhibitor identified by the present screening methods, the effect of the inhibition by such a compound results in inhibition of the nephropathy, or some related biochemical or physiologic aspect thereof, for example, podocyte morphology and/or proliferation, up- or down-regulation of gene expression and the like in the absence of the added candidate substance.

In other embodiments, one may seek compounds that actually augment the effects of Nef interaction with Src family tyrosine kinases.

iii. Candidate Substances

As used herein the term “candidate substance” refers to any molecule that may potentially act as an inhibitor of the present invention. The candidate substance may be a protein or fragment thereof, a small molecule inhibitor, or even a nucleic acid molecule. It may prove to be the case that the most useful pharmacological compounds will be compounds that are structurally related to other known modulators of HIV related nephropathy or other kidney disease, such as broad spectrum nucleoside reverse transcriptase inhibitors (e.g., Zidovudine) and other drugs commonly used in HAART, steroids such as prednisone and other corticosteroids, ramipril and the like (see Winston el al., Semin. Nephrol., 20(3):293-298, 2000 for review of treatments for HIVAN). Rational drug design includes not only comparisons with known inhibitors, but predictions relating to the structure of target molecules. Particularly useful compounds for use in rational drug design are those that will inhibit the interaction of Nef with the RT loop of the SH3 domain of Src family tyrosine kinases.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like Nef, or a fragment thereof. This could be accomplished by x-ray crystallography, computer modeling or by a combination of both approaches. Those of skill in the art are referred to Arold et al., 1997 (Structure, 5(10): 1361-1372) and Arold et al., 2001 (TIBS 26(6):356-3363) for a detailed description of the crystal structure of Nef and the relationship of the structure to the function of this HIV protein.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically—or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds molded of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of Src family tyrosine kinases, nephropathy or kidney other disease.

Other suitable inhibitors include antisense molecules, ribozymes, and antibodies (including single chain antibodies), each of which would be specific for a target located within the pathway activated by the interaction of Nef with a Src family tyrosine kinase. Such compounds are described in greater detail elsewhere in this document. For example, an antisense molecule that bound to a translational or transcriptional start site of Nef, or an antibody that bound to the C-terminus of Nef, would be ideal candidate inhibitors.

“Effective amounts” in certain circumstances are those amounts effective to reproducibly decrease podocyte dedifferentiation and/or proliferation of the cell and/or alter the expression of genes of the cell regulated by Nef in comparison to their normal levels. Compounds that achieve significant appropriate changes in activity will be used.

Significant changes in nephropathy, e.g., as measured in using podocyte growth, dedifferentiation, podocyte gene expression, kinase assays, and the like are represented by a alterations in activity of at least about 30%-40%, and most preferably, by changes of at least about 50%, with higher values of course being possible. The active compounds of the present invention also may be used for the generation of antibodies which may then be used in analytical and preparatory techniques for detecting and quantifying further such inhibitors.

b. In Vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to the Nef molecule or fragment thereof is provided.

The target may be either free in solution, fixed to support, expressed in or on the surface of a cell. Either the target or the compound may be labeled, thereby permitting determining of binding. In another embodiment, the assay may measure the inhibition of binding of a target to a natural or artificial substrate or binding partner (such as Nef and a member of the Src tyrosine kinase family). Competitive binding assays can be performed in which one of the agents (Nef, for example) is labeled. Usually, the target will be the labeled species, decreasing the chance that the labeling will interfere with the binding moiety's function. One may measure the amount of free label versus bound label to determine, binding or inhibition of binding.

A technique for high throughput screening of compounds is described in WO 94/03564. Large numbers of small peptide test compounds are synthesized on a solid substrate, such as plastic pins or some other surface. The peptide test compounds are reacted with, for example, Nef and washed. Bound polypeptide is detected by various methods.

Purified target, such as Nef, can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link an active region (e.g., the C-terminus of Nef) to a solid phase.

c. In Cyto Assays

Various cell lines that exhibit nephropathic characteristics can be utilized for screening of candidate substances. For example, podocyte cells containing engineered HIV-Nef, as discussed above, can be used to study various functional attributes of candidate compounds. In such assays, the compound would be formulated appropriately, given its biochemical nature, and contacted with a target cell.

Depending on the assay, culture may be required. As discussed above, the cell may then be examined by virtue of a number of different physiologic assays (growth, size, morphology etc). Alternatively, molecular analysis may be performed in which the function of Nef and related pathway may be explored. This involves assays such as those for protein expression, enzyme function, substrate utilization, mRNA expression (including differential display of whole cell or polyA RNA) and others.

For cell-based assays, an exemplary cell that may be used in the screening assays of the present invention is a conditionally-immortalized mouse renal podocyte cell line infected with either empty vector (control) or a Nef-expressing retrovirus (Nef expression confirmed by western blotting). Under non-permissive conditions for SV40 Tag-induced immortalization (37EC, no interferon), only the Nef-podocytes proliferate. In an exemplary assay, a multi-well format assay may be set up to determine cell proliferation of this cell line (monitored by e.g., using MTT reagent and spectrophotometric analysis) to identify compounds that inhibit Nef-specific proliferation. In such an assay, candidate substance that are non-specifically toxic should also inhibit control (vector) cell proliferation at the permissive temperature for Tag function (33EC, plus interferon).

For cell-free assays, Src family-Nef interaction can be assessed by using a solid-phase binding assay. GST-Hck SH3 protein expressed and purified from E. coli can be coated onto plastic wells in a multi-well format plate. Binding to biotinylated Nef peptide can be assessed using avidin-alkaline phosphatase plus an appropriate soluble colorimetric substrate. The inhibitory effects of a candidate inhibitory substance can be assessed by loss of the alkaline phosphatase substrate colorimetric readout as determined by spectrophotometric analysis.

d. In Vivo Assays

The present invention particularly contemplates the use of various animal models. Here, transgenic mice are contemplated and provide a specific model for HIVAN in a whole animal system. The generation of these animals has been described elsewhere in this document. These models can, therefore be used not only screen for inhibitors of the Nef/Src interaction but also to track the progression of nephropathic disease.

Treatment of these animals with test compounds will involve the administration of the compound, in an appropriate form, to the animal. Administration will bc by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

Determining the effectiveness of a compound in vivo may involve a variety of different criteria. Such criteria include, but are not limited to, survival, reduction of protein excretion, and improvement of general physical state including activity. It also is possible to perform histologic studies: on tissues from these mice, or to examine the molecular and morphological state of the cells, which includes cell size or alteration in the expression of nephropathy related genes.

H. Pharmaceutical Compositions

In the sections above, the present invention describes various novel compositions for the inhibition of the Nef/Src interaction, also described are assays for identifying additional composition. It is contemplated that therapeutic compositions of the present invention will be useful in the intervention of various disease states such as for example, HIVAN, AIDS dementia; HIV-induced anemia; HIV-induced lymphoma; HIV-induced myopathy; HIV-induced cardiomyopathy; and primary HIV-induced disease progression and any other disorders mediated through the interaction of HIV-1 Nef with Src family tyrosine kinases. Such agents may be used either alone or in combination with other therapeutic agents presently being used to control the deleterious effects of HIV-1 infection. In order to be used in such therapeutic indications, it will be preferable to prepare the compositions of the invention in pharmaceutically acceptable formats.

Also, it should be understood that it may well be that purified compositions that inhibit the interaction of Nef with a member of the Src family of tyrosine kinases may be routinely prepared into pharmaceutically acceptable forms of the proteins once they are isolated from the media and/or cellular compositions described above. Generally, this will entail preparing compositions that are essentially free of pyrogens, as well as other impurities that could be harmful to humans or animals.

One will generally desire to employ appropriate salts and buffers to render the compositions stable and allow for uptake by target cells. Buffers also will be employed when recombinant cells or nucleic acids are introduced into a subject. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic compositions produced by the present invention, its use in therapeutic compositions is contemplated. Supplementary active ingredients also can be incorporated into the compositions.

The compositions of the present invention include classic pharmaceutical preparations. Administration of these compositions according to the present invention will be via any common route so long as the target tissue is available via that route. The pharmaceutical compositions maybe introduced into the subject by any conventional method, e.g., by intravenous, intradermal, intramusclar, intramammary, intraperitoneal, intrathecal, intraocular, retrobulbar, intrapulmonary (e.g., term release); by oral, sublingual, nasal, anal, vaginal, or transdermal delivery, or by surgical implantation at a particular site, e.g., embedded under the splenic capsule, brain, or in the cornea. The treatment may consist of a single dose or a plurality of doses over a period of time.

The compositions produced using the present invention may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganism.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

For oral administration the compositions produced by the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. For example, parenteral administration may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042) pp 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood levels in conjunction with relevant dose-response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

It will be appreciated that the pharmaceutical compositions and treatment methods employing such compositions may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey, ducks and geese.

### EXAMPLES

The following examples present preferred embodiments and techniques. These examples are not intended to be limiting. Those of skill in the art will, in light of the present disclosure, appreciate that many changes can be made in the specific materials and methods which are disclosed and still obtain a like or similar result without departing from the spirit and scope of the invention.

### Example 1

### Materials and Methods

The present example provides details of materials and methods employed throughout the application and in the Examples presented herein below. This example provides specific reagents and conditions employed in particular assays and experiments exemplified below. However, those of skill in the art will be aware of other sources of reagents, other assays and conditions that may substitute for those exemplified in the present example.

Conditionally Immortalized Murine Podocyte Clones:

To isolate conditionally immortalized murine podocytes, heterozygous HIV-1 transgenic mice (“Tg26”, FVB/N, pNL4-3:d1443) described previously (Dickie et al., Virology 185:109-119, 1991; Kopp et al., Proc Natl Acad Sci USA 89:1577-1581, 1992) were bred with H-2K^{b}-tsA58 Immortamice™ (Charles River Laboratories, Wilmington, Mass.). F1 progeny were tested for the presence or absence of the HIV-1 transgene by Southern blot analysis as well as by PCR of genomic DNA. The immortalized podocytes were isolated from littermates that did not carry HIV-1 transgene (Mundel et al., Exp Cell Res 236:248-258, 1997; Schwartz et al., J Am Soc Nephrol 12:1677-1684, 2001).

The isolation of immortalized podocytes from HIV-1 negative mice was performed to match the genetic background to their HIV-1 positive littermates, which develop HIVAN. The cells were maintained in RPMI supplemented with 10% FBS, 100 U/ml penicillin, 100 μg/ml streptomycin, and 2 mM L-glutamine (Life Technologies, Rockville, Md.) at 33° C. in the presence of 5% CO₂. To permit immortalized growth, the medium was supplemented with 10U/ml recombinant mouse γ-interferon (Life Technologies, Rockville, Md.) to induce the H-2 K^{b} promoter driving synthesis of the temperature sensitive (tsA58) SV-40 T antigen and the cells were cultured at 33° C. (permissive conditions). To induce differentiation, cells were cultured on type I collagen at 37° C. without γ-interferon, resulting in degradation of the T-antigen (nonpermissive conditions).

Generation of Mutant HIV-1 Constructs Using pNL4-3 Provirus:

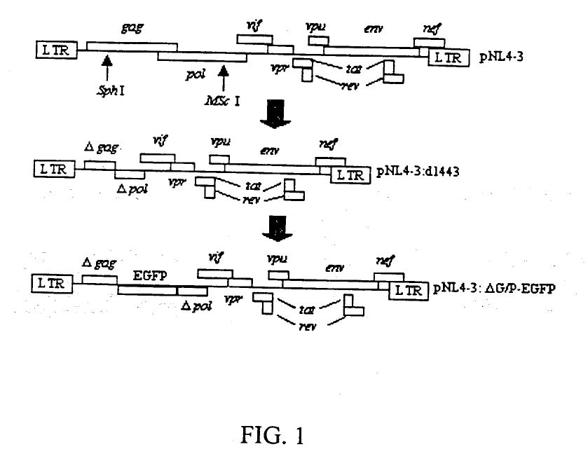

HIV-1 gag/pol-deleted pNL4-3:d1443 (Bruggeman et al., J. Clin. Invest. 100:84-92) was used as the parental construct in the present study with the following modifications. To monitor transduction of HIV-1 genes, a fragment containing the EGFP reporter gene (from pEGFP-C1; Clontech, Palo Alto, Calif.) was inserted in place of the gag/pol deletion in HIV-1 proviral construct, pNL4-3:d1443 (FIG. 1) (Kopp et al., Contrib Nephrol 107:194-204, 1994). The resulting construct (pNL4-3: ΔG/P-EGFP) was used to delineate the contribution of individual HIV-1 genes by mutating single or multiple genes. The mutations were made by in vitro site-directed mutagenesis using GenEditor (Promega, Madison, Wis.) or QuickChange mutagenesis kit (Stratagene, La Jolla, Calif.).

HIV-1 env and/or accessory genes (vif vpr, vpu, rev and nef) were individually mutated specifically by inactivating the start codon without affecting the reading frame of other viral proteins that utilize the same transcript (Table 2). All mutations were confirmed by sequencing, and all the mutated constructs were tested for loss of gene expression by western blotting (Ugen et al., In: Vaccine 93, Cold Spring Harbor. Cold Spring Harbor Laboratories, pp 215-221, 1993; Goncalves et al, J Virol 68:704-712, 1994; Shugars et al., J Virol 67:4639-4650, 1993; Maldarelli et al., J Virol 67:5056-5061, 1993). Western blots, however, revealed that the construct mutated for Vif and another construct mutated for Nef could utilize downstream ATG codons. Therefore, vif was further mutated by altering the downstream ATG codons at positions 5086 and 5125. The nef downstream ATG was interrupted by digesting with Xho I, filling in with Klenow enzyme and religating with T4 DNA ligase (New England Biolabs, Beverly, Mass.). Each subsequent construct of Vif and Nef mutations showed loss of expression by western blot analysis. To explore a role for Tat and Rev, Tat and Nef, or Tat alone, these genes were left intact for expression while all the remaining genes were mutated in the vector backbone (Table 2).

Cloning of Single HIV-1 Genes in Retroviral Expression Vectors:

Individual HIV-1 genes (env, vif, vpr, vpu, tat, and nef) were cloned into pHR-CMV-IRES2-GFP-ΔB vector (a gift of Dr. James C. Mulloy, Memorial Sloan Kettering Cancer Center, New York, N.Y.). The genes were amplified by PCR using premix Taq (TaKaRa Biomedicals, PanVera Corporation, Madison, Wis.) and cloned at Bam HI-Sal I site or Bam HI-Eco RI sites of the vector. The correct orientation of the gene was checked by sequencing and the expression was confirmed by western blot analysis. In spite of repeat cloning of Nef and examining each time that it was cloned in correct orientation with correct sequences, it was not expressed in podocytes. Therefore, Nef was cloned at Bam HI-Sal I site of the pBabe-puro retroviral expression vector (Morgenstern et al., Nucleic Acids Res 18:3587-3596, 1990). The expression of these single gene constructs was confirmed by western blot analysis.

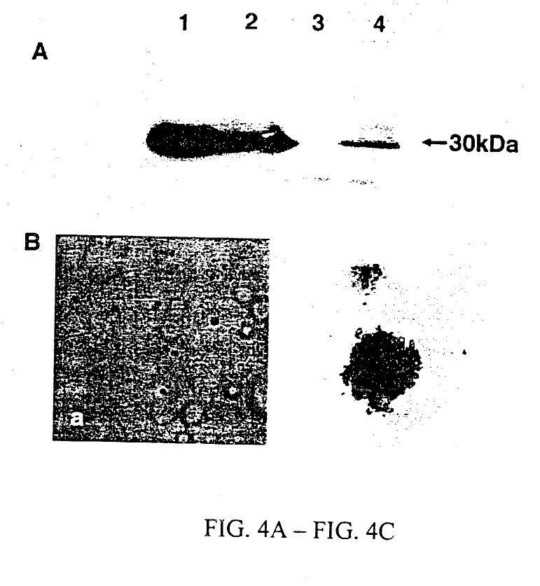

The Nef was expressed in podocytes by this vector as seen by western blotting (FIG. 4A through FIG. 4B).

Production of Pseudotyped Retroviral Supernatant:

The HIV-1 parental construct (pNL4-3: ΔG/P-EGFP), mutated HIV-1 plasmid constructs and single HIV-1 gene constructs were used to produce VSV.G pseudotyped viruses to provide pleiotropism and high titer virus stocks. Infectious viral supernatants were produced by transient transfection of 293T cells using Lipofectamine 2000 (Life Technologies, Rockville, Md.) according to the manufacturer's instructions. The HIV-1 gag/pol and VSV.G envelop genes were provided in trans using pCMV R8.91 and pMD.G plasmids, respectively (gifts of Dr. Didier Trono, Salk Institute, La Jolla, Calif.) (Naldini et, al., Science 272:263-267, 1996). The Moloney murine leukemia virus gag/pol genes were provided using REP/GP plasmid to produce pseudotyped virus from pBabe-puro. As a negative control, virus was also produced from pHR-CMV-IRES2-GFP-ΔB which contained HIV-1 LTRs and EGFP as well as pBabe-puro empty expression vectors. The viral stocks were titrated by infecting 293T cells with 10-fold serial dilutions. The reciprocal of the lowest dilution showing expression of green fluorescence protein (GFP) was defined as the transducing units/ml. The transduction efficiency of cells infected with MOI of 1-5 was monitored for GFP expression by fluorescence microscopy. Depending upon the titer of the virus, 50-80% of cells showed GFP expression at day 5. Viral stocks ranging from 10⁵-10⁸ infectious virus units per ml were obtained. Some low titer viral stocks were further concentrated by ultracentrifugation.

Transduction of Podocytes and Soft Agar Analysis:

The podocytes at a concentration of 50,000 per plate were seeded in a 60-cm dish in the medium described above but without IFNγ. Next day, the cells were washed twice with serum free RPMI 1640 and then infected with MOI of 1-5 infectious virus units in the presence of 5 μg/ml polybrene. At day 7, the cells were trypsinized and approximately 40,000 cells were suspended in 0.3% soft agar containing 1×RPMI, 1×PenStrep, 10 mM Hepes, pH 7.0, 10% FBS and 13.2 mM NaHCO₃ and then plated on a 6-cm dish followed by incubation at 33° C. for 4 weeks. Every 5 days, 1.0 ml media was replenished.

Cell Growth Assay

Podocytes transduced with pBabe-puro/nef or the empty pBabe-puro vector were grown in the presence of puromycin (0.5 μg/ml) under permissive conditions at a density of 10,000 cells per well in 1.0 ml growth medium at 33° C. The cells were counted using a hemocytometer after trypan blue dye exclusion at 3-day intervals for 15 days: To inactivate T-antigen, cells were cultured for 7 days under nonpermissive conditions without puromycin before they were plated in collagen-coated 6-well plates at 20,000 cells/well. Podocytes were further cultured for 7 or 14 days under nonpermissive conditions in the absence of puromycin. Triplicate wells were trypsinized and counted.

Cell Culture of Mouse Podocytes and RNA Isolation

After virus infection, podocytes were cultured under permissive conditions on collagen-coated plates until they reached confluence. Podocytes transduced with pBabe-puro/nef or pBabe-puro vector were selected with puromycin (0.5 μg/ml) under permissive conditions on collagen-coated plates until they reached confluence. They were cultured under nonpermissive conditions without puromycin for 14 days, at which time photographs were taken and total RNA was extracted using Trizol (Life Technologies).

Northern Blot Analysis

Probes were obtained by RT-PCR of RNA isolated from glomeruli of a normal mouse. Probes were derived from the following published cDNA sequences: synaptopodin (Gene Bank accession number, NM021695, nucleotide positions 642-1343), podocalyxin (AF109393, 4313-4991), CALLA (M81591, 1454-2151), Ki-67 (X82786, 7588-8286), cyclin A (Z26580, 1035-1740), cyclin E (X75888, 500-1200), p21 (U24173, 93-394), p27 (U09968, 138-628), p57 (NM009876, 580-1315), WT-1 (M55512, 1416-1898), G3PDH (NM008084, 566-1017), ezrin (X60671, 2039-2680). The cDNA probes were radiolabeled with [³²P-α] dCTP by random oligonucleotide priming. Expression levels were quantified by UN-SCAN-IT (Silk Scientific Corporation, Orem, Utah).

Microarray Analysis

The Atlas Mouse cDNA Expression Array (Clontech Laboratories), which contains 588 mouse cDNAs was used in the present study. Total RNA was isolated from podocytes transduced with nef or vector alone. Microarray analysis was performed according to manufacturer's instructions. cDNA expression levels were quantified by UN-SCAN-IT. These signals were normalized to five house-keeping gene controls, including G3PDH, beta-actin, ribosomal protein S29, ubiquitin, and ornithine decarboxylase. Only those genes with signal differences greater than 2-fold were considered as modulated.

Kinase Assays

Cells were cultured under nonpermissive conditions for 14 days. Following lysis in RIPA buffer, the lysates (500 μg) were incubated with polyclonal anti-Hck antibody (Santa Cruz Biotechnology, Calif.) and protein A beads, or with mouse monoclonal anti-Src antibody 327 (Parsons, et al., J. Virol. 59:755-758) and anti-mouse antibody prebound to protein A/G beads. The immunocomplex was washed twice with RIPA buffer and twice with kinase buffer (20 mM HEPES [pH 7.4], 10 mM MgCl₂, containing 10 μg of enolase (Sigma)) and then resuspended in 15 μl of kinase buffer and 25 μCi of [γ-32P]ATP. After 20 min of incubation at 32° C., the reaction was stopped by adding 30 μl of 2× protein loading dye. This mixture was boiled and electrophoresed through an SDS-10% polyacrylamide gel, transferred to Immobilon-P membrane (Millipore Corp., Bedford, Mass.), followed by autoradiography.

Western Blot Analysis

RIPA lysates (25 μg) were separated by SDS-polyacrylamide gel electrophoresis and transferred to Immobilon-P membrane. The filter was blocked with 5% skim milk, 0.05% Tween-20 in PBS, and then incubated with anti-Src antibody 327 (1:2000) or anti-Hck antibody (1:400), and horseradish peroxidase-conjugated secondary antibodies. The filter was visualized with Supersignal (Pierce, Rockford, Ill.).

### Example 2

### Nef Induces Anchorage-Independent Growth of Podocytes

The HIV-1 provirus, pNL4-3 was modified by deleting 3108 bp region of gag/pol using digestion enzymes MSc I and Sph I and substituting it with EGFP gene. The new construct pNL4-3: ΔG/P-EGFP (FIG. 1) was used as parental construct and the following genes, env or one of the accessory genes (vif, vpr, vpu, nef, or rev) was mutated to abolish their expression in order to screen their precise role in podocytes proliferation. Each mutated gene showed lack of expression as confirmed by western blot analysis. Once it was confirmed that mutated gene was not expressed, the construct was used to produce viral supernatant.

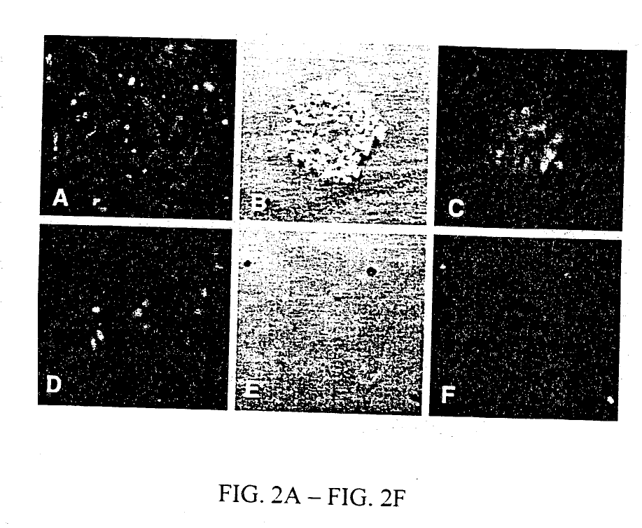

After determining the viral titer, the virus was inoculated to infect podocyte cells. The infection efficiency was monitored by GFP expression under fluorescence microscopy (Olympus 1×70). Depending upon the titer of the virus, up to 80% of the cells showed GFP expression at day 5 (FIG. 2A-FIG. 2F.) Western blotting of the podocyte lysate was performed again to rule out recombination or reversion of the mutated gene. The podocytes infected with pBabe-puro/nef were selected by adding 1.5 μg/ml of puromycin antibiotic (Sigma-Aldrich, Inc.) in the growth medium and the expression of Nef was observed by western blotting (FIG. 4A and FIG. 4B). Thereafter, the infected podocytes were analyzed for anchorage-independent growth in soft agar.

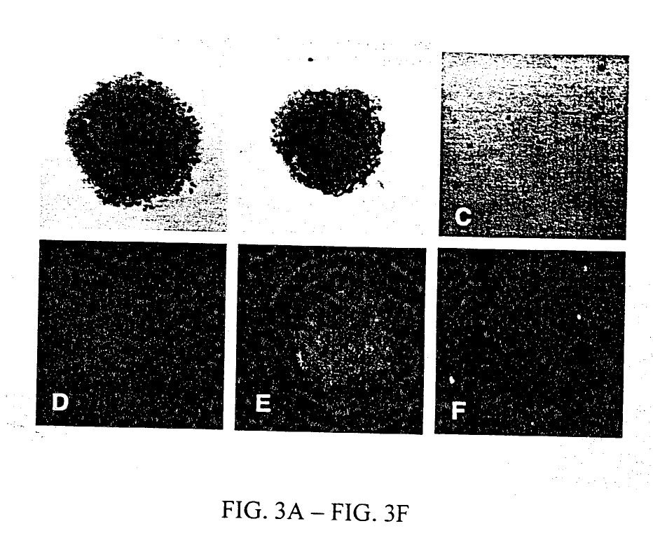

There is a high frequency of colony formation in the cells infected with parental virus (FIG. 2A through FIG. 2F) or viruses deleted for any one of Env, Vif, Vpr, Vpu or Rev (FIG. 3A through FIG. 3F), suggesting colony forming activity is independent of these genes. In contrast, very few colonies were observed with HR-CMV-IRES2-EGFP control virus (FIG. 2A through FIG. 2F) and nef-deleted viruses (FIG. 3A through FIG. 3F), which indicates that Nef is necessary for colony formation in soft agar.

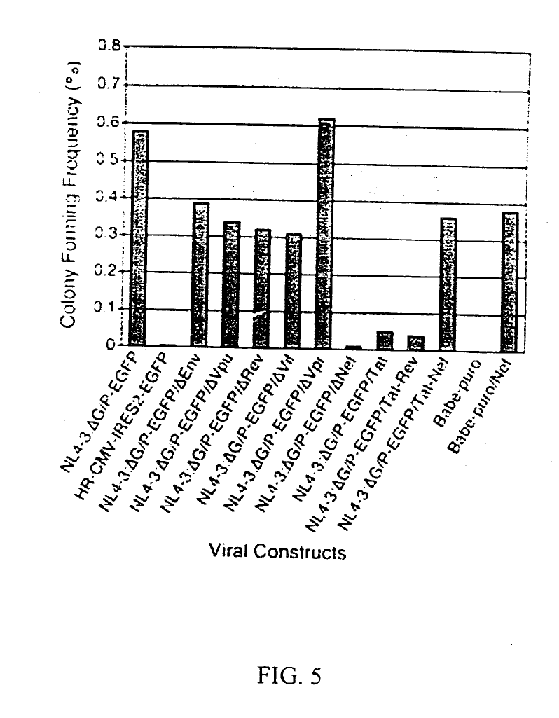

Since Tat is essentially required for LTR derived transcription, all the above deletion constructs contained functional Tat. Therefore, identical experiments were performed using viruses expressing Tat alone, Tat and Rev or Tat and Nef (Table 2). No significant colony formation was observed with the expression of Tat alone or Tat and Rev, whereas strong colony formation was observed when Nef was co-expressed with Tat i.e., Tat and Nef (FIG. 5). These experiments demonstrate that except Nef, all the other HIV-1 genes have very little or no role in induction of growth of podocytes in soft agar.

TABLE 2
Various mutated constructs made by altering initiation codon of env and/or
accessory genes in pNL4-3: ΔG/P-EGFP parental construct

| | | | Start ATG |
|---|---|---|---|
| Viral Construct | Mutated Gene(s) | Expressed Gene(s) | mutated to |
| pNL4-3: ΔG/P- | — | env, vif, vpr, vpu, | — |
| EGFP | | tat rev, and nef | |
| pNL4-3: ΔG/P- | env | vif, vpr, vpu, tat | ACg |
| EGFP/ΔEnv | | rev, and nef | |
| pNL4-3: ΔG/P- | vif | env, vpr, vpu, tat, | TGA |
| EGFP/ΔVif | | rev, and nef | |
| pNL4-3: ΔG/P- | vpr | env, vif, vpu, tat, | Gtg |
| EGFP/ΔVpr | | rev and nef | |
| pNL4-3: ΔG/P- | vpu | env, vif, vpr, tat, | TGA |
| EGFP/ΔVpu | | rev and nef | |
| pNL4-3: ΔG/P- | nef | env, vif, vpr, vpu, | TGA |
| EGFP/ΔNef | | tat, and rev | |
| pNL4-3: ΔG/P- | rev | env, vif, vpr, vpu, | ACA |
| EGFP/ΔRev | | tat and nef | |
| pNL4-3: ΔG/P- | env, vif, vpr, vpu | tat and rev | |
| EGFP/Tat-Rev | and nef | | |
| pNL4-3: ΔG/P- | env, vif, vpr, vpu | Tat | |
| EGFP/Tat | rev and nef | | |
| pNL4-3: ΔG/P- | env, vif, vpr, vpu | tat and nef | |
| EGFP/Tat-Nef | and rev | | |

To determine conclusively if Nef alone was sufficient to induce anchorage-independent growth, podocyte infection was repeated using virus expressing Nef under Moloney murine leukemia virus (MMLV) LTR (pBabe-puro/Nef) and then assayed the cells in soft agar. FIG. 4A shows that the podocytes infected with the MMLV based vector expressed the Nef about five-fold less than that expressed by the HIV-1 parental construct. These cells, however, showed potent colony forming activity (FIG. 4A through FIG. 4B). FIG. 5 shows the quantification of colony formation in soft agar.

Since podocytes were conditionally immortalized, the anchorage-independent growth might be affected by the HIV-induced upregulation of T antigen. No increase in T antigen expression was found either in the presence or absence of Nef expression.

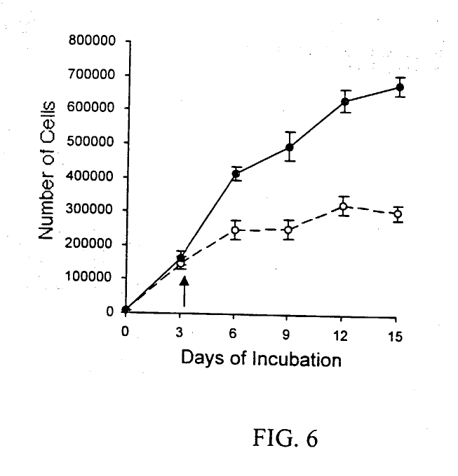

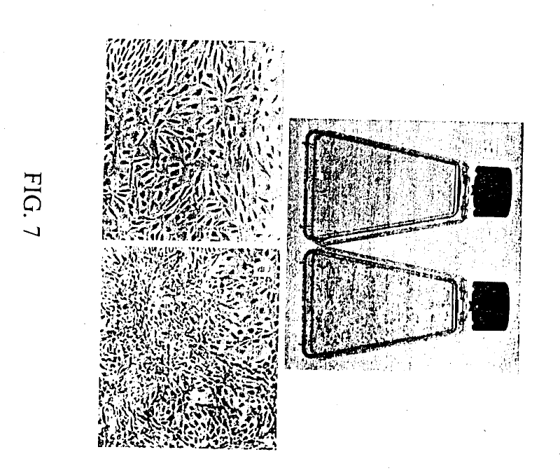

Viable cell count by trypan blue dye exclusion revealed that the cells infected with pBabe-Puro/Nef construct and pBabe-Puro vector alone grow in cell culture almost at the same rate until they reach to confluence. Once the cells are confluent, the podocytes containing empty vector are contact inhibited and no significant increase in cell number is observed on longer incubation whereas in the presence of Nef, podocytes continue to proliferate after confluence showing a statistically significant difference in growth (P<0.001) (FIG. 6). The increased growth in culture was visually evident when the cells showed focus formation in Nef infected podocytes and absence of foci without Nef, which is a clear indication of loss of contact inhibition induced by Nef (FIG. 7).

### Example 3

### HIV-1 Dysregulates Podocyte Gene Expression In Vitro

Infection of podocytes with HIV-1 pNL4-3 virus induces podocyte proliferation and a loss of contact inhibition (Schwartz, et al., J. Am. Soc. Nephrol. 12:1677-1684, 2001). Since HIVAN lesions show a loss of several markers of podocyte differentiation, studies were performed to determined whether in vitro infection of podocytes had similar effects on proliferation and differentiation markers on differentiated cells. The data from these studies are presented in the present example.

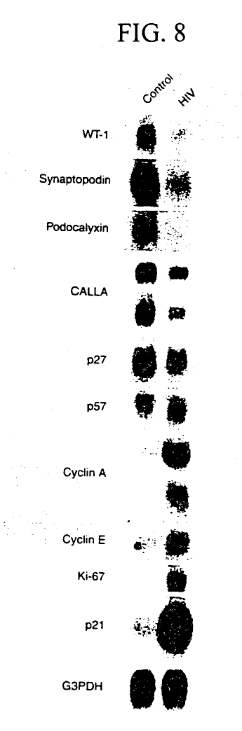

Conditionally immortalized murine podocytes were transduced with HIV-1 pNL4-3:d1443 and cultured under non-permissive conditions for 14 days. Northern blot analysis revealed that the expression of WT-1, synaptopodin, podocalyxin, CALLA and the cyclin-dependent kinase inhibitor p27 was downregulated, and the expression of Ki-67, cyclin-dependent kinase inhibitor p21, and cyclin A was up-regulated in HIV-1 transduced podocytes compared to mock-transduced cells (FIG. 8). These changes in gene expression are similar to what is detected in collapsing glomerulopathy (Barisoni et a., J. Am. Soc. Nephrol. 10:51-61, 1999; Barisoni et al., Kidney Int. 58:137-143, 2000; Barisoni, et al., Kidney Int. 58:173-181, 2000; Shankland et al., Kidney Int. 58:674-683, 2000). Expression of cyclin dependent kinase inhibitor p57 remained unchanged. As a result, the in vitro system was used to investigate which of the HIV-1 genes may be responsible for these changes.

### Example 4

### Mapping of HIV-1 Gene Responsible for Gene Dysregulation

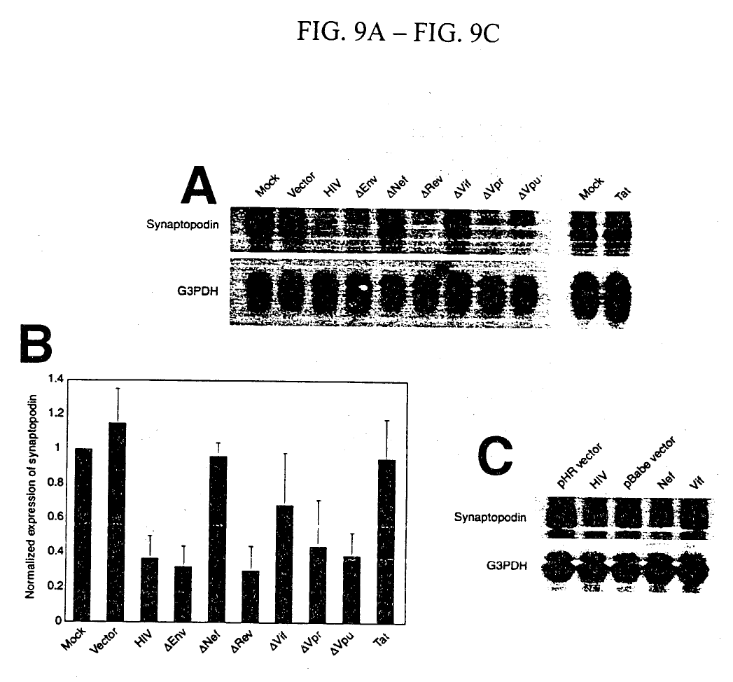

Synaptopodin, an actin-associated protein which is expressed in normal podocytes and is lost in collapsing glomerulopathy in HIVAN and in idiopathic collapsing glomerulopathy (Barisoni et al., J. Am. Soc. Nephrol, 10:51-61, 1999), was used as a marker of podocyte differentiation. To map the HIV-1 genes responsible for the downregulation of synaptopodin, stop codons were introduced into the parental constructs to prevent expression of Env, Nef, Rev, Vif, Vpr, or Vpu. HIV-1 Tat is required for transcriptional transactivation from the HIV-LTR in the clones used in these studies, therefore, it was not mutated. Instead tat was cloned into pHR-CMV-IRES2-GFP-ΔB vector to access its individual effects. Downregulation of synaptopodin occurred in cells infected with HIV-1 pNL4-3 and with viruses deficient in Env, Rev, Vpr or Vpu, suggesting that these gene products were not necessary for synaptopodin inhibition. Similarly when Tat was expressed as a single gene construct, there was no downregulation of synaptopodin. In contrast, viruses deficient in Nef or Vif did not inhibit synaptopodin expression (FIG. 9A). The quantitation of the results in FIG. 9A is shown in FIG. 9B. Because the expression level of HIV-1 genes was not equal in each of the mutant viruses, the data could not reveal which of the two HIV-1 genes, nef or vif, is responsible for decreased expression of synaptopodin. Thus, podocytes were transduced with single gene constructs containing either vif or nef. Whereas Nef caused potent downregulation of synaptopodin, Vif alone had no effect on synaptopodin expression (FIG. 9C). Although Nef is clearly responsible for this effect, it may be that Vif plays a modulating or regulatory role in the context of virus infection.

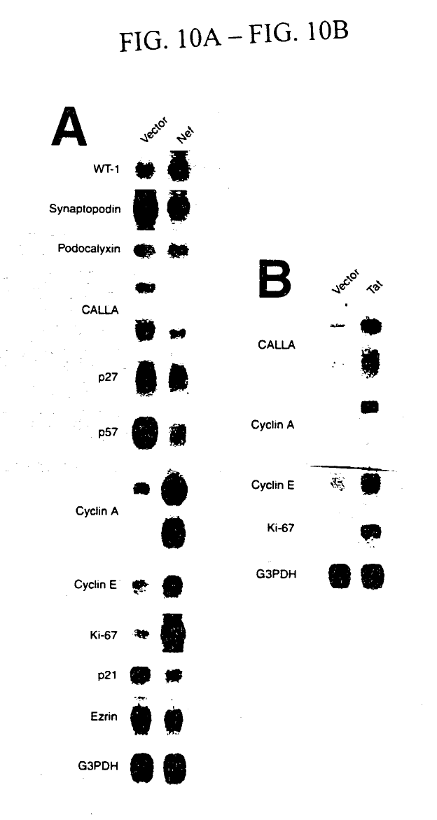

In addition, the effect of single gene constructs (Nef or Tat alone) on expression levels of other genes was investigated by northern blot analysis, and revealed that constructs containing Nef or Tat recapitulated many of the findings with HIV-1 pNL4-3. The expression of CALLA, p27 and cyclin-dependent kinase inhibitor p57 was decreased, while the expression of Ki-67, cyclin A and cyclin E, which are markers of proliferation, was increased by the presence of Nef (FIG. 10A and FIG. 10B). WT-1 and podocalyxin showed no change. Decreased expression was seen with ezrin and p21. Tat also upregulated the expression of Ki-67, cyclin A, and cyclin E (FIG. 10B), however, it did not decrease the expression levels of CALLA (FIG. 10B) or synaptopodin (FIG. 9A). This suggests that Tat has a role in HIV-1 induced podocyte proliferation, whereas Nef is involved in both proliferation and dedifferentiation.

### Example 5

### Nef Promotes Proliferation and Induces a Loss of Contact Inhibition of Podocytes

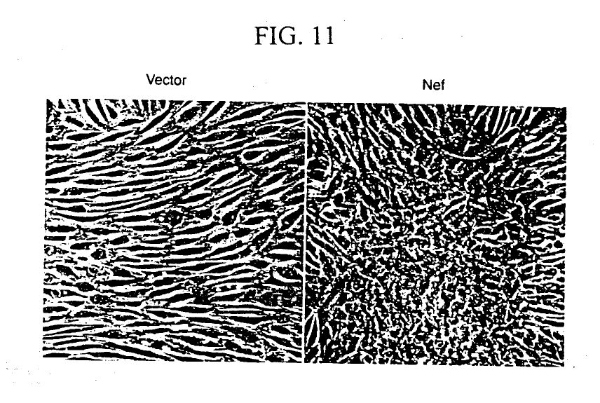

Data presented above in Example 2 shows that Nef induces anchorage-independent growth of podocytes as observed by colony formation in soft agar under permissive conditions. Since podocytes do not form colonies in soft agar under non-permissive conditions (37° C.), HIV-induced morphological changes were examined on collagen-coated plates at 37° C. Podocytes transduced with vector alone formed a flat monolayer, while podocytes transduced with Nef overgrew the monolayer, forming foci typical of contact independent growth (FIG. 11). In addition, Nef-transduced podocytes exhibited a rounded morphology, akin to the morphology of podocytes isolated from HIV-1 transgenic mice (Schwartz, et al., J. Am. Soc. Nephrol. 12:1677-1684, 2001), thereby demonstrating that these changes were not due to clonal variation but to HIV-1 gene expression, specifically Nef expression.

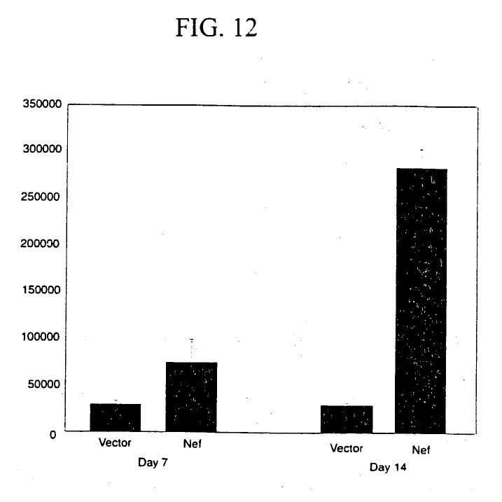

The effect of Nef on podocyte proliferation is shown in FIG. 12. Nef enhanced the proliferation of podocytes on day 7 (2.5-fold increase), more significantly on day 14 (tenfold increase).

### Example 6

### Differential Gene Expression to Identify Candidate Genes Involved in Podocyte Dysregulation

To identify candidate genes involved in podocyte dysregulation as a result of HIV-1 Nef expression, gene expression profiles from Nef or vector transduced podocytes were compared. Results of quantification are summarized in Table 3. Of a total of 588 well-defined cDNAs, 6 genes were upregulated and 22 genes were downregulated in Nef-transduced podocytes compared to controls. Consistent with the northern analysis shown here, p57 was found to be downregulated nearly 4-fold by Nef. Ezrin, which is expressed in normal podocytes and diminishes in an early stage of podocyte injury (Hugo et al., Kidney Int. 54:1934-1944, 1998), was decreased by Nef. The expression of PCNA, clusterin, and b-Raf was increased in Nef-transduced podocytes. The increased expression was reported to be associated with proliferation and nodule formation in several cell lines (Millis et al., J. Cell. Physiol. 186: 210-219, 2001; Dugan et al., J. Biol. Chem. 274: 25842-25848, 1999). In contrast, the expression of p57, hepatocyte nuclear factor 3, pur-alpha, CTCF, c-erb A, and Tob was decreased by Nef. The association between decreased expression of these genes and increased proliferation was also reported in various cell lines (Nakamura, et al, Biochem. Biophys. Res. Commun. 253:352-357, 1998; Stacey et al., Oncogene 18:4254-4261, 1999; Rasko et al., Cancer Res. 61:6002-6007, 2001. Iglesias et al., Cell Growth Differ. 5:697-704, 1994; Matsuda et al., Oncogene 12:705-713, 1996).

TABLE 3
Summary of differentially expressed genes in pBabe-puro/nef vs. pBabe-
puro vector-transduced podocytes. 6 genes were expressed more than
two-fold higher in Nef-transduced podocytes. 22 genes were
downregulated more than two-fold in Nef-transduced podocytes

| | Fold change |
|---|---|
| Genes upregulated by Nef | |
| Hox-2.5 | 2.9 |
| Clusterin | 2.8 |
| cyclin B2 | 2.6 |
| PCNA | 2.6 |
| HMG-14 chromosomal protein | 2.3 |
| B-Raf proto-oncogene | 2 |
| Genes downregulated by Nef | |
| Cek 5 receptor protein tyrosine kinase ligand | 4 |
| Cyclin dependent kinase inhibitor p57 | 3.9 |
| interleukin-5 receptor | 3.6 |
| Nucleobindin | 3.6 |
| Heat shock transcription factor 1 | 3.5 |
| erythrocyte glucose transporter-1 (GLUT-1) | 3 |
| monocyte chemoattractant protein 1 receptor (CCR2) | 3 |
| hepatocyte nuclear factor 3 | 3 |
| pur-alpha | 2.6 |
| CTCF | 2.6 |
| UBF | 2.6 |
| Ski proto-oncogene | 2.4 |
| Sp4 transcription factor | 2.4 |
| transforming growth factor beta | 2.3 |
| xeroderma pigmentosum group B complementing protein | 2.2 |
| (XPB) | |
| cyclin B1 | 2.2 |
| Integrin beta | 2.1 |
| Egr-1 | 2.1 |
| c-erb A | 2.1 |
| Tob (Transducer of ErbB-2) | 2 |
| xeroderma pigmentosum group G complementing protein | 2 |
| (XPG) | |
| granulocyte-macrophage colony stimulating factor receptor | 2 |

### Example 7

### Nef Activates Src Tyrosine Kinase in Podocytes

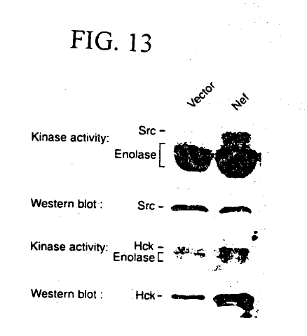

Previous studies have shown that Nef binds to SH3 domains of Src kinase family members and promotes their transforming activities (reviewed in refs. 19). The expression of Src family members was examined by RT-PCR and immunoprecipitation. These investigations revealed that that Hck, Lyn, Lck, and Src were expressed in podocytes transduced with Nef or vector. To explore a possible role for these kinases in Nef-induced loss of contact inhibition in podocytes, the specific activity of several Src tyrosine kinases was determined. Src and Hck were immunoprecipitated from podocytes transduced with Nef or vector, and then incubated with [γ-32P]-ATP and enolase. As shown in FIG. 13, the specific activity of the Src tyrosine kinase was increased 2.3-fold in cells expressing Nef. The expression level of Src protein showed no change with Nef by western blotting. In contrast, both the expression and activity level of Hck increased in Nef-expressing cells. These data suggest that Nef induces Src and Hck activities in podocytes.

Numerous modifications and variations of the present invention are possible in light of the above teachings and, therefore, are within the scope of the invention. The entire disclosure of all publications cited herein are hereby incorporated by reference,

What is claimed is:

1. A method of inhibiting kidney cell dedifferentiation, comprising inhibiting the interaction of Nef with a Src family tyrosine kinase SH3 domain of a polypeptide of said cell.

2. The method of claim 1, wherein said cell is located in vitro.

3. The method of claim 1, wherein said cell is located in vivo.

4. The method of claim 1, wherein said Nef is HIV-1 Nef.

5. The method of claim 1, wherein said inhibiting the interaction of Nef with a SH3 domain of a Src family tyrosine kinase comprises reducing the expression of Nef in said cell.

6. The method of claim 1, wherein said inhibiting the interaction of Nef with a SH3 domain of a Src family tyrosine kinase comprises contacting said Nef with an agent that binds to and/or inactivates said Nef.

7. The method of claim 5, wherein said method comprises contacting said cell with a nucleic acid construct that reduces the expression of Nef in said cell.

8. The method of claim 6, wherein said agent is a peptide inhibitor comprising a variant of the PXXP motif of the SH3 binding domain of Nef.

9. The method of claim 8, wherein said peptide inhibitor comprises a sequence selected from the group consisting of SEQ ID NO:1, SEQ ID NO:2, SEQ ID NO:3, SEQ 4, SEQ ID NO:5, SEQ ID NO: 6, SEQ ID NO:7, SEQ ID NO:8, SEQ ID NO:9, SEQ ID NO:10, SEQ ID NO:1, SEQ ID NO:12, SEQ ID NO:13, SEQ ID NO:14, and SEQ ID NO:15.

10. The method of claim 6, wherein said agent is a small molecule antagonist of the SH3 binding domain of Nef.

11. The method of claim 6, wherein said agent is a peptidomimetic antagonist of the SH3 binding domain of Nef.

12. The method of claim 6, wherein said agent is an anti-Nef antibody preparation.

13. The method of claim 12, wherein said antibody preparation comprises a single chain antibody.

14. The method of claim 12, wherein said antibody is a monoclonal antibody.

15. The method of claim 14, wherein said antibody binds the PXXP motif of the SH3 binding domain of HIV-1 Nef.

16. The method of claim 1, wherein said kidney cell is a podocyte.

17. A transgenic non-human animal, wherein a podocyte of said animal comprises an HIV-1 Nef gene under the control of a kidney cell-specific promoter.

18. The transgenic non-human animal of claim 17, wherein the specific activity of a Src family tyrosine kinase in the podocyte of the transgenic animal is increased relative to the Src tyrosine kinase activity level of a podocyte from a wild-type animal of the same species.

19. The transgenic non-human animal of claim 17, wherein the expression of one or more nucleic acids selected from the group consisting of Cek 5 receptor protein tyrosine kinase ligand; Cyclin dependent kinase inhibitor p57; interleukin-5 receptor; nucleobindin; Heat shock transcription factor 1; erythrocyte glucose transporter-1 (GLUT-1); monocyte chemoattractant protein 1 receptor (CCR2); hepatocyte nuclear factor 3; pur-alpha; CTCF; UBF; Ski proto-oncogene; Sp4 transcription factor; transforming growth factor beta; xeroderma pigmentosum group B complementing protein (XPB); cyclin B1; Integrin beta; Egr-1; c-erbA; Tob (Transducer of ErbB-2); xeroderma pigmentosum group G complementing protein (XPG); and granulocyte-macrophage colony stimulating factor receptor is decreased in a podocyte of the transgenic animal relative to a podocyte from a wild-type animal of the same species.

20. The transgenic non-human animal of claim 17, wherein the expression of one or more nucleic acids selected from the group consisting of Hox-2.5; clusterin; cyclin B2; PCNA; HMG-14 chromosomal protein; and B-Raf proto-oncogene is increased in a podocyte of the transgenic animal relative to a podocyte from a wild-type animal of the same species.

21. The transgenic animal of claim 17, wherein said promoter is a nephrin promoter.

22. The transgenic animal of claim 17, wherein said promoter is a CX promoter.

23. A recombinant host cell, wherein said cell is transformed with an expression construct comprising a nucleic acid that encodes HIV-1 Nef under the control of a kidney cell-specific promoter.

24. The recombinant host cell of claim 23, wherein said cell is a podocyte.

25. The recombinant host cell of claim 23, wherein said promoter is a nephrin promoter.

26. The recombinant host cell of claim 23, wherein said expression construct comprises a Nef sequence from pNL4-3 contained in GenBank Accession # AF324493 (nucleotides 8787 to 9407).

27. A method for screening for agents that modulate nephropathy comprising:
a) providing a cell expressing HIV-1 Nef;
b) contacting said cell with a candidate modulator; and
c) monitoring said cell for change in a cellular property associated with nephropathy that occurs in the presence of said modulator.

28. The method of claim 27, wherein said cell is a kidney cell.

29. The method of claim 27, wherein said cell is a podocyte.

30. The method of claim 29, wherein said cell is a primary podocyte.

31. The method of claim 30, wherein said primary podocyte is derived from a subject having HIVAN.

32. The method of claim 27, wherein said contacting is performed in vitro.

33. The method of claim 27, wherein said contacting is performed in vivo.

34. The method of claim 33, wherein said cell is part of a transgenic, non-human animal.

35. The method of claim 34, wherein protein excretion of said animal is monitored.

36. The method of claim 27, wherein said monitoring comprises monitoring the specific activity of Src family tyrosine kinases of said cell in the presence and absence of said candidate modulator.

37. The method of claim 27, wherein said monitoring comprises determining the expression of more one or more nucleic acids selected from the group consisting of Cek 5 receptor protein tyrosine kinase ligand; Cyclin dependent kinase inhibitor p57; interleukin-5 receptor; nucleobindin; Heat shock transcription factor 1; erythrocyte glucose transporter-1 (GLUT-1); monocyte chemoattractant protein 1 receptor (CCR2); hepatocyte nuclear factor 3; pur-alpha; CTCF; UBF; Ski proto-oncogene; Sp4 transcription factor; transforming growth factor beta; xeroderma pigmentosum group B complementing protein (XPB); cyclin B1; Integrin beta; Egr-1; c-erbA; Tob (Transducer of ErbB-2); xeroderma pigmentosum group G complementing protein (XPG); granulocyte-macrophage colony stimulating factor receptor; Hox-2.5; clusterin; cyclin B2; PCNA; HMG-14 chromosomal protein; and B-Raf proto-oncogene in the presence and absence of said candidate modulator.

38. The method of claim 27, wherein said candidate modulator is a nucleic acid construct that reduces the expression of Nef.

39. The method of claim 27, wherein said candidate modulator is an antibody.

40. The method of claim 39, wherein said candidate modulator is a single chain antibody.

41. The method of claim 39, wherein said candidate modulator is a monoclonal antibody.

42. The method of claim 39, wherein said monoclonal antibody binds the PXXP motif of the SH3 binding domain of HIV-1 Nef.

43. A composition comprising a candidate modulator of nephropathy identified according to a method of any one of claims 27 through 42.

44. A peptide composition comprising a sequence selected from the group consisting of the peptide sequences described in Table 1 and Table 1A.

45. A method of treating a subject, comprising inhibiting the interaction of Nef with a SH3 domain of a Src family tyrosine kinase, wherein said subject has a disease associated with HIV-1 infection.

46. The method of claim 45, wherein said disease is a HIV-induced disease selected from the group consisting of HIV associated nephropathy (HIVAN) AIDS dementia; anemia; lymphoma; myopathy; cardiomyopathy; and primary HIV-induced disease progression.

47. The method of claim 45, wherein said inhibiting comprises administering a composition of claim 43.

48. The method of claim 45, wherein said inhibiting comprises administering a composition of claim 44.
